SEQUENCE LISTING (8 sequences)

SEQ ID NO: 1 - DNA, 20 bp, synthetic construct
  source 1..20: mol_type = other DNA
agrgttygatymtggctcag

SEQ ID NO: 2 - DNA, 19 bp, synthetic construct
  source 1..19: mol_type = other DNA
rgytaccttgttacgactt

SEQ ID NO: 3 - DNA, 20 bp, synthetic construct
  source 1..20: mol_type = other DNA
caatggrsgvrasyctgahs

SEQ ID NO: 4 - DNA, 20 bp, synthetic construct
  source 1..20: mol_type = other DNA
aaactyaaarraattgacgg

SEQ ID NO: 5 - DNA, 15 bp, synthetic construct
  source 1..15: mol_type = other DNA
yaacgagcgmraccc

SEQ ID NO: 6 - DNA, 15 bp, synthetic construct
  source 1..15: mol_type = other DNA
gggtykcgctcgttr

SEQ ID NO: 7 - DNA, 17 bp, synthetic construct
  source 1..17: mol_type = other DNA
attaccgcggctgctgg

SEQ ID NO: 8 - DNA, 19 bp, synthetic construct
  source 1..19: mol_type = other DNA
tgctgcctcccgtaggagt

(19) United States
(12) Patent Application Publication
(10) Pub. No.: US 2026/0234544 A1
(43) Pub. Date: Aug. 13, 2026

(54) MICROALGAE GROWTH-PROMOTING MICROORGANISM AND MICROALGAE GROWTH PROMOTER

(71) Applicants: Kankyo Daizen Co., Ltd., Kitami-shi Hokkaido (JP); National University Corporation Hokkaido Higher Education and Research System, Obhiro-shi Hokkaido (JP)

(72) Inventors: Masaaki Konishi, Kitami-shi Hokkaido (JP); Yuta KATO, Kitami-shi Hokkaido (JP); Tomomi Kusano, Kitami-shi Hokkaido (JP); Pei Yu Tan, Kitami-shi Hokkaido (JP); Makoto Kubonouchi, Kitami-shi Hokkaido (JP)

(73) Assignees: Kankyo Daizen Co., Ltd., Kitami-shi Hokkaido (JP); National University Corporation Hokkaido Higher Education and Research System, Obihiro-shi Hokkaido (JP)

(21) Appl. No.: 19/259,837

(22) Filed: Jul. 3, 2025

Related U.S. Application Data
(63) Continuation of application No. PCT/JP2023/045712, filed on Dec. 20, 2023.

(30) Foreign Application Priority Data
Jan. 6, 2023 (JP) 2023-000957
Sep. 28, 2023 (JP) 2023-166726

(51) Int. Cl. C12N 1/20; C12N 1/12; C12R 1/10

(52) U.S. Cl. CPC C12N 1/20; C12N 1/12; C12R 2001/10

(57) ABSTRACT

This microorganism belongs to the genus Rhodococcus, Xanthobacter, Ancylobacter, Shewanella, or Aeromonas. Said microorganism may be Rhodococcus cerastii belonging to the genus Rhodococcus, Xanthobacter flavus belonging to the genus Xanthobacter, or Ancylobacter rudongensis belonging to the genus Ancylobacter. Said microorganism may be for promoting the growth of cyanobacterium, green algae, glaucophyte algae, or Euglena algae. Said microorganism may be for promoting the growth of organisms belonging to the phylum Cyanobacteria, Ochrophyta, Euglena, Cryptophyta, Haptophyta, Cercozoa, Glaucophyta, Rhodophyta, Chlorophyta, or Streptophyta.

### CROSS-REFERENCE TO RELATED APPLICATIONS

The present application is a continuation application of International Application number PCT/JP2023/45712, filed on Dec. 20, 2023, which claims priority under 35 U.S.C § 119(a) to Japanese Patent Application No. 2023-000957, filed on Jan. 6, 2023 and Japanese Patent Application No. 2023-166726, filed on Sep. 28, 2023 contents of which are incorporated herein by reference in their entirety.

### SEQUENCE-LISTING

This application contains a Sequence Listing which has been submitted electronically in XML format and is hereby incorporated by reference in its entirety. The Sequence Listing XML file, created on Jan. 5, 2023, is named SequenceList.xml and is 7,781 bytes in size.

### TECHNICAL FIELD

The present invention relates to a microalgae growth-promoting microorganism that promotes the growth of microalgae, a microalgae growth promoter, a microalgae culture method using the microalgae growth-promoting microorganism, and a screening method for isolating microorganisms that can promote the growth of microalgae.

### BACKGROUND OF THE INVENTION

For example, microalgae are used in the industrial production of useful substances such as astaxanthin, which is one type of naturally derived carotenoid pigment that exhibits a red color. Useful substances are extracted from microalgae, and used as raw materials of food, pharmaceuticals, feed, fertilizers, and the like. Recently, using sugars and lipids accumulated within the cells of microalgae as useful substances, and producing petroleum and bioethanol have been attracting attention. In addition, Euglena sp., which is one type of microalgae, is itself industrially produced as a raw material for food. Microalgae can fix carbon dioxide by their photosynthetic capability. Because of this, culturing microalgae is also useful as a global warming countermeasure.

The growth rate of microalgae is slower than typical heterotrophic microorganism. For example, Japanese Unexamined Patent Application Publication (Translation of PCT Application) No. 2014-509188 discloses that the growth rate of microalgae is raised by increasing the levels of nutrients such as nitrogen and phosphorus in order to enhance the economic efficiency of producing useful substances using microalgae and the like. However, there is a problem with the method described in Japanese Unexamined Patent Application Publication (Translation of PCT Application) No. 2014-509188 that the growth rate of microalgae cannot be increased sufficiently.

### BRIEF SUMMARY OF THE INVENTION

The present invention has been made in view of these matters, and an object thereof is to provide a microalgae growth-promoting microorganism that can promote the growth of microalgae, a microalgae growth promoter containing the microorganism, a microalgae culture method, and a screening method for isolating microorganisms that can promote the growth of microalgae.

A microalgae growth-promoting microorganism according to a first aspect of the present invention is a microorganism belonging to Rhodococcus, Xanthobacter, Ancylobacter, Shewanella, Bacillus, or Aeromonas.

A microalgae growth promoter according to a second aspect of the present invention contains the microalgae growth-promoting microorganism.

### BRIEF DESCRIPTION OF THE DRAWINGS

FIG. 1 depicts the concentration of each medium component added to an Ormerod medium.

FIG. 2 depicts the concentration of each medium component of a co-culture medium.

FIG. 3 depicts primers used in PCR reactions.

FIG. 4 depicts isolates whose chlorophyll fluorescence intensity ratios on day 6 of co-culture exceeded 1.0.

FIG. 5 depicts isolates whose chlorophyll fluorescence intensity ratios on day 6 of co-culture exceeded 1.0.

FIGS. 6A and 6B depict temporal changes in chlorophyll a during the monoculture of PCC7942 and during the co-culture with the AF2108 strain.

FIG. 7 depicts the results of flow cytometry measurements of the cell count, cell size, and chlorophyll fluorescence intensity at 168 hours of culture time during the monoculture of PCC7942 and during the co-culture with the AF2108 strain.

FIGS. 8A through 8G depict, in chronological order, the state of monoculture of PCC7942 and co-culture with the AF2108 strain in flasks.

FIGS. 9A and 9B depict temporal changes in chlorophyll a+b during the monoculture of NIES-2173 and during the co-culture with the AF2108 strain.

FIG. 10 depicts the results of flow cytometry measurements of the cell count, cell size, and chlorophyll fluorescence intensity at 120 hours of culture time during the monoculture of NIES-2173 and during the co-culture with the AF2108 strain.

FIG. 11 depicts the phylogenetic tree of Rhodococcus.

FIGS. 12A and 12B depict temporal changes in chlorophyll a during the monoculture of PCC7942 and during the co-culture with the AF2111 strain.

FIG. 13 depicts the results of flow cytometry measurements of the cell count, cell size, and chlorophyll fluorescence intensity at 168 hours of culture time during the monoculture of PCC7942 and during the co-culture with the AF2111 strain.

FIGS. 14A through 14G depict, in chronological order, the state of monoculture of PCC7942 and co-culture with the AF2111 strain in flasks.

FIGS. 15A and 15B depict temporal changes in chlorophyll a+b during the monoculture of NIES-2173 and during the co-culture with the AF2111 strain.

FIG. 16 depicts the results of flow cytometry measurements of the cell count, cell size, and chlorophyll fluorescence intensity at 120 hours of culture time during the monoculture of NIES-2173 and during the co-culture with AF2111.

FIG. 17 depicts the phylogenetic tree of Xanthobacter.

FIGS. 18A and 18B depict temporal changes in chlorophyll a during the monoculture of PCC7942 and during the co-culture with the GA1226 strain.

FIG. 19 depicts the results of flow cytometry measurements of the cell count, cell size, and chlorophyll fluorescence intensity at 168 hours of culture time during the monoculture of PCC7942 and during the co-culture with the GA1226 strain.

FIGS. 20A through 20G depict, in chronological order, the state of monoculture of PCC7942 and co-culture with the GA1226 strain in flasks.

FIGS. 21A and 21B depict temporal changes in chlorophyll a+b during the monoculture of NIES-2173 and during the co-culture with the GA1226 strain.

FIG. 22 depicts the results of flow cytometry measurements of the cell count, cell size, and chlorophyll fluorescence intensity at 120 hours of culture during the monoculture of NIES-2173 and during the co-culture with the GA1226 strain.

FIG. 23 depicts the phylogenetic tree of Ancylobacter.

FIGS. 24A and 24B depict temporal changes in chlorophyll a during the monoculture of PCC7942 and during the co-culture with the OR151 strain.

FIG. 25 depicts the results of flow cytometry measurements of the cell count, cell size, and chlorophyll fluorescence intensity at 168 hours of culture time during the monoculture of PCC7942 and during the co-culture with the OR121 strain.

FIGS. 26A through 26G depict, in chronological order, the state of monoculture of PCC7942 and co-culture with the OR151 strain in flasks.

FIGS. 27A and 27B depict temporal changes in chlorophyll a+b concentration during the monoculture of NIES-2173 and during the co-culture with the OR151 strain.

FIG. 28 depicts the results of flow cytometry measurements of the cell count, cell size, and chlorophyll fluorescence intensity at 120 hours of culture during the monoculture of NIES-2173 and during the co-culture with the OR151 strain.

FIG. 29 depicts the phylogenetic tree of Shewanella.

FIG. 30 depicts the composition of a JCM medium.

FIG. 31 depicts the composition of a modified CM medium.

FIG. 32 depicts some of isolates obtained in an implementation example.

FIG. 33 depicts temporal changes in the chlorophyll amount during monoculture or co-culture.

FIG. 34 depicts the results of flow cytometry.

FIG. 35 depicts the phylogenetic tree of Bacillus closely related to JM311.

FIG. 36 depicts temporal changes in the chlorophyll amount during monoculture or co-culture.

FIG. 37 depicts the results of flow cytometry.

FIG. 38 depicts the phylogenetic tree of Bacillus closely related to JM321.

FIG. 39 depicts temporal changes in the chlorophyll amount during monoculture or co-culture.

FIG. 40 depicts the results of flow cytometry.

FIG. 41 depicts the phylogenetic tree of Rhodococcus closely related to AF2108.

FIG. 42 depicts temporal changes in the chlorophyll amount during monoculture or co-culture.

FIG. 43 depicts the results of flow cytometry.

FIG. 44 depicts the phylogenetic tree of Aeromonas closely related to JM202.

### DETAILED DESCRIPTION OF THE INVENTION

Hereinafter, the present disclosure will be described through exemplary embodiments, but the following exemplary embodiments do not limit the invention according to the claims, and not all of the combinations of features described in the exemplary embodiments are necessarily essential to the solution means of the invention.

[Microalgae Growth-Promoting Microorganism]A microalgae growth-promoting microorganism according to the present embodiment is a microorganism belonging to Rhodococcus (Rhodococcus), Xanthobacter (Xanthobacter), Shewanella (Shewanella), Ancylobacter (Ancylobacter), Bacillus (Bacillus), or Aeromonas (Aeromonas). The microorganism is one obtained through screening using fermented cattle urine (Fermented Cattle Urine, FCU), which is obtained by processing cattle urine with microorganisms, and a microalgae growth promoter produced from FCU, and the microalgae growth-promoting microorganism according to the present embodiment is not limited to ones isolated from FCU as long as the microalgae growth-promoting microorganism is a microorganism that belongs to any of the genera described above, and acts on microalgae to promote their growth. Preferably, the microalgae growth-promoting microorganism is Rhodococcus cerastii (Rhodococcus cerastii) of Rhodococcus, Xanthobacter flavus (Xanthobacter flavus) of Xanthobacter, Ancylobacter rudongensis (Ancylobacter rudongensis) of Ancylobacter, or Shewanella sp. (Shewanella sp.).

In addition, preferably, the microalgae growth-promoting microorganism is Bacillus licheniformis (Bacillus licheniformis), Bacillus pumilus (Bacillus pumilus), Bacillus zhangzhouensis (Bacillus zhangzhouensis), Peribacillus acanthi (Peribacillus acanthi), Bacillus australimaris (Bacillus australimaris), or Bacillus safensis (Bacillus safensis) of Bacillus, Rhodococcus cerastii (Rhodococcus cerastii) of Rhodococcus, or Aeromonas salmonicida (Aeromonas salmonicida) or Aeromonas piscicola (Aeromonas piscicola) of Aeromonas. The microalgae growth-promoting microorganism according to the present embodiment can promote the growth of microalgae by co-culturing with the microalgae or adding or mixing a medium, in which the microalgae growth-promoting microorganism is cultured, to or with a medium of the microalgae.

### [Microalgae]

The microalgae growth-promoting microorganism according to the present embodiment promotes the growth of microalgae. The microalgae are a type of photosynthetic organism that require a microscope to identify individual bodies.

For example, the microalgae are species belonging to Phylum Cyanobacteria (e.g. blue-green algae), Heterokontophyta, Euglenophyta (e.g. Euglena algae), Cryptophyta, Haptophyta, Cercozoa, Glaucophyta (e.g. glaucophyte algae), Rhodophyta, Chlorophyta (e.g. green algae), or Streptophyta. The microalgae are preferably organisms belonging to Phylum Cyanobacteria such as blue-green algae or organisms belonging to Euglenophyta such as Euglena algae.

For example, examples of the microalgae include species belonging to Chroococcales, Oscillatoriales, Nostocales, or Stigonemales of Phylum Cyanobacteria, and specifically examples thereof include Chroococcus sp. (Chroococcus sp.), Microcystis aeruginosa (Microcystis aeruginosa), Oscillataria sp. (Oscillataria sp.), Microcoleus sp. (Microcoleus sp.), Nostoc sp. (Nostoc sp.), Cylindrospermum sp. (Cylindrospermum), Stigonema sp. (Stigonema), and Synecochoccus elongatus (Synechococcus elongatus).

For example, the microalgae may be species belonging to Ochromonadales or Mallomonadales of Chrysophyceae of Heterokontophyta, and specifically examples thereof include Uroglenopsis americana (Uroglenopsis americana), Uroglena volvox (Uroglena volvox), Mallomonas (Mallomonas), and Synura sp. (Synura sp.). For example, the microalgae may be species belonging to Bacillariophyceae of Heterokontophyta, and specifically examples thereof include Coscinodiscus sp. (Coscinodiscus sp.) and Diatoma (Diatoma).

For example, the microalgae may be species belonging to Xanthophyceae of Heterokontophyta, and specifically examples thereof include Pseudostaurastrum sp. (Pseudostaurastrum sp.) and Characiopsis sp. (Characiopsis sp.). For example, the microalgae may be species belonging to Dictyochophyceae of Heterokontophyta, and specifically examples thereof include Dictyocha sp. (Dictyocha sp.). For example, the microalgae may be species belonging to Dinophyceae of Heterokontophyta, and specifically examples thereof include Peridinium sp. (Peridinium sp.) and Scrippsiella trochoidea (Scrippsiella trochoidea).

For example, the microalgae may be species belonging to Euglenophyceae of Euglenophyta, and specifically examples thereof include Euglena sp. (Euglena sp.) and Phacus sp. (Phacus sp.). For example, the microalgae may be species belonging to Cryptophyceae of Cryptophyta, and specifically examples thereof include Cryptomonas sp. (Cryptomonas sp.) and Rhodomonas sp. (Rhodomonas sp.).

For example, the microalgae may be species belonging to Haptophyceae of Haptophyta, and specifically examples thereof include Coronosphaera sp. (Coronosphaera sp.) and Gephyrocapsa sp. (Gephyrocapsa sp.). For example, the microalgae may be species belonging to testate filose amoebae of Cercozoa, and specifically examples thereof include Paulinella chromatophora (Paulinella chromatophora). For example, the microalgae may be species belonging to Glaucophyceae of Glaucophyta, and specifically examples thereof include Glaucocystis sp. (Glaucocystis sp.).

For example, the microalgae may be species belonging to Rhodophyceae of Rhodophyta, and specifically examples thereof include Cyanidium sp. (Cyanidium sp.) and Galdieria sp. (Galdieria sp.). For example, the microalgae may be species belonging to Chlorophyceae of Chlorophyta, and specifically examples thereof include Pediastrum duplex (Pediastrum duplex), Volvox sp. (Volvox sp.), Chlamydomonas sp. (Chlamydomonas sp.), Asterococcus sp. (Asterococcus sp.), and Chlorella. For example, the microalgae may be species belonging to Trebouxiophyceae of Chlorophyta, and specifically may be unicellular green algae belong to Chlorella. For example, the unicellular green algae belonging to Chlorella may be Chlorella sorokiniana (Chlorella sorokiniana).

For example, the microalgae may be species belonging to Mesostigmatophyceae of Streptophyta, and specifically examples thereof include Mesostigma sp. (Mesostigma sp.). For example, the microalgae may be species belonging to Conjugatophyceae of Streptophyta, and specifically examples thereof include Zygnema sp. (Zygnema sp.). For example, examples of the microalgae include Synecochoccus elongatus (Synechococcus elongatus) of Phylum Cyanobacteria, which is a type of so-called blue-green algae.

### [Microalgae Growth Promoter]

The microalgae growth promoter containing the microalgae growth-promoting microorganism according to the present embodiment is not limited in form. The microalgae growth promoter may be liquid, solid, slurry, or the like, and is preferably liquid. In addition, the microalgae growth promoter according to the present embodiment may contain secondary components such as, for example, diluents, stabilizers, thickeners, and granulating agents depending on its form. For example, the microalgae growth promoter can be used by being added to a medium for culturing microalgae.

The microalgae growth promoter according to the present embodiment contains the microalgae growth-promoting microorganism mentioned above. The microalgae growth promoter may be used as is, or the microorganism may be removed therefrom before use. The method for removing the microorganism is not particularly restricted, and, for example, examples thereof include removal by filtering, removal by centrifugation, sterilization using an autoclave, and sterilization by ultraviolet ray irradiation. The microalgae growth promoter according to the present embodiment can promote the growth of microalgae by being added to a medium for culturing the microalgae.

A microalgae culture method according to the present embodiment includes co-culturing the microalgae promoting microorganism mentioned above with the microalgae. For example, the microalgae culture method according to the present embodiment includes co-culturing, with the microalgae, the microalgae promoting microorganism in an FCU medium. In this manner, the microalgae culture method according to the present embodiment can promote the growth of the microalgae.

### [Screening Method]

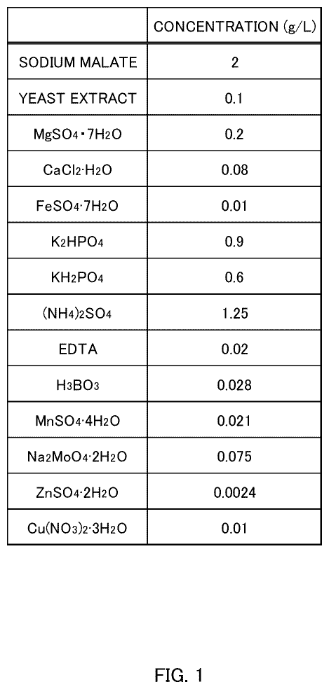

A screening method according to the present embodiment is a method of screening microorganisms capable of promoting the growth of microalgae, and includes the following steps (1) and (2).
(1) A step of co-culturing a screening-target microorganism with microalgae.
(2) A step of quantifying chlorophyll contained in the medium after the co-culture.
   The method of preparing the screening-target microorganism in (1) is not limited particularly. For example, a candidate microorganism can be isolated from a naturally derived liquid that can promote the growth of microalgae like fermented cattle urine (Fermented Cattle Urine, FCU), which is fermented by aerating cattle urine. The screening method according to the present embodiment may treat, as the screening target, a microorganism contained in a livestock urine fermentation liquid obtained by fermenting urine of other livestock such as horses or pigs, instead of cattle urine. In a case where FCU is used, the screening method according to the present embodiment includes culturing an Ormerod medium by adding a diluent solution obtained by diluting the FCU. FIG. 1 depicts the concentration of each medium component added to an Ormerod medium. The screening method according to the present embodiment includes repeating an operation of isolating a colony formed in an Ormerod medium to a second Ormerod medium, and culturing the colony in the second Ormerod medium to obtain a plurality of single colonies. The screening method according to the present embodiment includes co-culturing, in a plurality of media including microalgae, each of the plurality of single colonies that have been formed.

In addition, the screening method according to the present embodiment may include culturing a screening-target microorganism in an FCU medium instead of an Ormerod medium or a JCM520 medium. In this case, the screening method according to the present embodiment may use FCU sterilized by autoclaving at 121° C. for 20 minutes, or sterilized by passing through a 0.22-μm filter. The screening method according to the present embodiment includes mixing FCU and sterile water to adjust the amount of FCU such that the concentration of FCU in the completed FCU medium becomes 20% or 10%. The screening method according to the present embodiment includes adding, in the FCU medium, gellan gum adjusted such that its concentration in the completed FCU medium becomes 0.8% or agar adjusted such that its concentration in the completed FCU medium becomes 2%.

The step of quantifying chlorophyll as depicted in (2) mentioned above includes quantifying chlorophyll contained in microalgae after the co-culture of a screening-target microorganism and the microalgae to enable an evaluation of the extent to which the microorganism has exhibited its microalgae growth-promoting effect. The step of quantifying chlorophyll includes using a fluorescence spectrophotometer to measure the fluorescence intensity at an excitation wavelength of 488 nm and a fluorescence wavelength of 683 nm to 720 nm. In addition, the step of quantifying chlorophyll can include changing the wavelengths depending on the type of microalgae. For example, the step of quantifying chlorophyll preferably includes measuring the fluorescence intensity at an excitation wavelength of 488 nm and a fluorescence wavelength of 683 nm in a case where the microalgae are PCC7972, at an excitation wavelength of 488 nm and a fluorescence wavelength of 685 nm in a case where the microalgae are NIES-2173, and at an excitation wavelength of 488 nm and a fluorescence wavelength of 700 nm in a case where the microalgae are NIES-48. In this manner, by measuring light at a wavelength corresponding to the fluorescence of chlorophyll, the step of quantifying chlorophyll enables an accurate evaluation of the extent to which a microorganism has promoted the growth of the microalgae.

The screening method according to the present embodiment includes decoding, through sequencing, the DNA sequence corresponding to 16S rRNA and the like in the microorganism genome of an identified colony. For example, the screening method according to the present embodiment includes identifying a known microorganism with the DNA sequence having the highest similarity to the decoded DNA sequence as the microorganism corresponding to the colony or a candidate for the microorganism corresponding to the colony. In addition, the screening method according to the present embodiment may include identifying a known microorganism with the DNA sequence having a similarity equal to or higher than a threshold to the decoded DNA sequence corresponding to 16S rRNA or the like in the microorganism genome of an identified colony as the microorganism corresponding to the colony. For example, the threshold is a value in the range from 97% to 98%.

### IMPLEMENTATION EXAMPLES

### [Microalgae Growth-Promoting Microorganism Isolation and Culture Methods]

In order to examine the screening method according to the present embodiment, the present inventors used an Ormerod medium to isolate a microorganism having the microalgae growth-promoting effect. The present inventors added 2% agar in addition to each medium component depicted in FIG. 1, and implemented autoclaving at 121° C. for 20 minutes to prepare an Ormerod agar medium. The present inventors used FCU having been fermented as the isolation source, and prepared a liquid in which the isolation-source FCU was diluted in the range of ten times to 10⁵ times. The present inventors sowed 100 μL of the diluted FCU onto the solidified Ormerod medium in each petri dish, inoculated the diluted FCU using a Conradi rod, put lids on the petri dishes, and cultured microorganisms in an environment under aerobic conditions, at room temperature (24° C.), with 115 to 120 μmol/m²/s light exposure for 12 hours out of every 24 hours until colonies were formed. The present inventors used a disposable loop to pick up the formed colonies, subcultured the formed colonies several times onto a medium similar to the medium used for the isolation, and obtained a single colony.

### [Summary of Screening of Microalgae Growth-Promoting Bacteria by High-Throughput Co-Culture Evaluation System]

### (1) Cyanobacteria

The present inventors used Synechococcus elongatus PCC7942 (hereinafter also referred to as PCC7942 simply), which is a model organism of blue-green algae, in order to evaluate the microalgae growth-promoting effect. In the case of the monoculture of PCC7942, as a preculture, the present inventors used a co-culture medium to perform flask culture at 30° C. and 120 rpm with a photon flux density of 115 to 120 μmol/m²/s under continuous light exposure for 24 hours for seven days. The photon flux density represents the intensity of light.

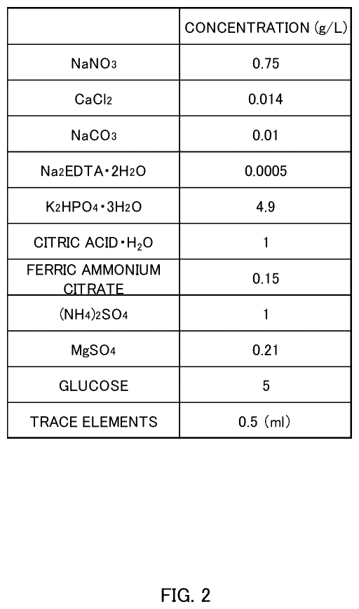

FIG. 2 depicts the concentration of each medium component of a co-culture medium. Trace elements in FIG. 2 that are contained in 100 mL of solution are 0.25 g of CuSO₄·5H₂O, 0.37 g of (NH₄)6Mo₇O₂₄·4H₂O, 2.47 g of H₃BO₃, 0.29 g of ZnSO₄·7H₂O, and 1.58 g of MnCl₂·4H₂O. The present inventors inoculated PCC7942 onto a co-culture medium without mixing PCC7942 with isolates, and performed main culture at 30° C. and 120 rpm with a photon flux density of 115 to 120 μmol/m²/s under continuous light exposure for 24 hours for seven days. The present inventors measured the chlorophyll a concentration (μg/mL) at 24-hour intervals.

In a case where isolates isolated from FCU and PCC7942 are co-cultured, as a preculture for the co-culture, the present inventors used a 100-mL flask, inoculated 500 μL of strain glycerol stock solution in 40 mL of co-culture medium, and co-cultured microorganisms at 30° C. and 160 rpm. During the main culture, the present inventors dispensed 40 mL of co-culture medium into a 100-mL flask, adjusted the amount of PC7942 such that the light absorbance A₇₃₀ at a wavelength of 730 nm of PCC7942 became 0.05 after mixing with isolates, and adjusted the amount of each isolate isolated from FCU such that the light absorbance A₆₀₀ at a wavelength of 600 nm of the isolate became 0.01, 0.05, 0.08, or the like after mixing with PCC7942.

The present inventors inoculated PCC7942 onto a co-culture medium after mixing PCC7942 and isolates, and performed main culture at 30° C. and 120 rpm with a photon flux density of 115 to 120 μmol/m²/s under continuous light exposure for 24 hours for seven days. The present inventors quantified the chlorophyll a concentration (μg/mL) at 24-hour intervals. The present inventors confirmed that each isolate did not exhibit chlorophyll fluorescence similar to the chlorophyll fluorescence of PCC7942. This demonstrates that each isolate does not affect the measurement results of chlorophyll fluorescence intensity derived from PCC7942 during the co-culture with PCC7942.

### <Chlorophyll Quantification>

The present inventors recovered 1 mL of sample after the co-culture, and centrifuged the sample at 15,000×g for seven minutes. The present inventors removed the supernatant fraction, and resuspended cells, recovered as a pellet, in 1 mL of chilled 100% (vol/vol) methanol. In order to extract pigments from cells, the present inventors put the sample in a dark location at 4.0° C. for one hour, and incubated the sample. After the incubation, the present inventors centrifuged the sample at 15,000×g for ten minutes at 4.0° C., and quantified chlorophyll a contained in the supernatant by spectrophotometry. The present inventors calibrated using methanol as a blank, and measured the absorbance at 665 nm and 720 nm. The present inventors determined the chlorophyll a concentration using the formulae (chlorophyll a concentration (μg/mL)=12.9447×(A₆₆₅−A₇₂₀)).

### <Flow Cytometry Analysis>

The present inventors measured the cell count and the chlorophyll fluorescence per cell using the flow cytometer Cube8. The present inventors prepared 1 mL of each culture solution diluted 1000 times, and treated them as measurement samples. The present inventors set the voltages for forward scatter (FSC-H), side scatter (SSC-H), and chlorophyll fluorescence (FL2-H) to 200.0 V, 275.0 V, 525.0 V, and 675.0 V, respectively.

### (2) Chlorella

### <Culture Method>

The present inventors evaluated the microalgae growth-promoting effect using, as another example of microalgae, Chlorella sorokiniana (Chlorella sorokiniana) NIES-2173 (hereinafter also referred to as NIES-2173 simply). The present inventors performed preculture in a modified BG11 medium in which 5 g/L glucose was added, for three days at 30° C., 125 rpm, PPFD, and 133 μmol/m²/s with a light-dark cycle of 24 h/0h using flask culture. In the case of isolates, for preculture, the present inventors used a 100-mL flask, inoculated 1 mL of strain glycerol stock solution in 40 mL of co-culture medium, and cultured microorganisms at 30° C. and 168 rpm.

During the main culture, the present inventors dispensed 40 mL of modified BG11 medium in which 5 g/L glucose was added to 100-mL flasks, made adjustments such that Chlorella sorokiniana NIES-2173 became A₇₅₀:0.025, and each strain became A₆₀₀:0.005, A₆₀₀: 0.025, A₆₀₀: 0.04, and the like, inoculated the mixture into the modified BG11 medium in which 5 g/L glucose was added, and cultured the mixture at 30° C., 125 rpm, PPFD, and 133 μmol/m²/s with a light-dark cycle of 24 h/0 h for four days. (n=3).

### <Chlorophyll Quantification>

The present inventors recovered 1 mL of culture solution into a 1.5-mL tube, and centrifuged the culture solution at 8,000 rpm for 10 min. at 4° C. After removing the supernatant, the present inventors added 1.5 mL of pure methanol, and left the cell pellet immersed in methanol at 4° C. in a dark location for 24 h. After 24 hours, the present inventors agitated the cell pellet using a vortex mixer, and centrifuged the cell pellet again at 8,000 rpm for 10 min at 4° C. The present inventors transferred the supernatant to a glass cuvette, and measured the absorbance at 653 nm, 666 nm, and 750 nm. The present inventors calculated the pigment content of chlorophyll a using a computation formula (1) ChlA (mg/L)=15.65(Abs666-Abs750)-7.34(Abs653-Abs750). In the computation formula (1), ChlA is the abbreviation for chlorophyll a. Abs653, Abs666, and Abs750 are the absorbance at wavelengths of 653 nm, 666 nm, and 750 nm, respectively. The present inventors calculated the pigment content of chlorophyll b using a computation formula (2) ChlB (mg/L)=27.05(Abs653-Abs750)-11.21(Abs666-Abs750) (2). In the computation formula (2), ChlB is the abbreviation for chlorophyll b.

### <Flow Cytometry Analysis>

The present inventors measured the absolute bacterial count and the chlorophyll fluorescence per cell using the flow cytometer Cube8. The present inventors prepared 1 mL of each culture solution diluted 100 times, and treated them as measurement samples. The present inventors set the voltages for forward scatter (FSC-H), side scatter (SSC-H), and chlorophyll fluorescence (FL2-H) to 125.0 V, 180.0 V, 525.0 V, and 400.0 V, respectively.

### [Genome Extraction of Microalgae Growth-Promoting Strain]

The present inventors conducted monoculture of each isolate and subjected it to genome extraction. The present inventors centrifuged a tube containing bacterial cells in a refrigerated centrifuge at 10,000 rpm and 4° C. for five minutes, and recovered a cell pellet (precipitate). The present inventors discarded the supernatant into a waste reservoir using a pipette. The present inventors put 560 μL of TE buffer into the cell pellet, thoroughly agitated the mixture, and suspended the cells. The present inventors added 30 μL of 10% SDS and 10 μL of proteinase K solution, thoroughly mixed the mixture, and then kept the mixture warm at 37° C. for one hour. The present inventors added 100 μL of 5M NaCl, and thoroughly mixed the mixture. The present inventors added 80 μL of CTAB/NaCl solution, thoroughly mixed the mixture, and kept the mixture warm at 65° C. for ten minutes. The present inventors put in 0.7 mL of chloroform/isoamyl alcohol, closed the lid of the tube, inverted the tube upside down five to six times, thoroughly agitated the mixture, and then centrifuged the mixture in a refrigerated centrifuge at 15,000 rpm for five minutes at 4° C.

The present inventors collected 0.5 to 0.6 mL of the upper layer of the liquid, and transferred the collected liquid to a new 1.5-mL microtube. The present inventors added an equal amount of phenol/chloroform/isoamyl alcohol to the transferred liquid amount, thoroughly agitated the mixture, and then centrifuged the mixture in a refrigerated centrifuge at 15,000 rpm for five minutes at 4° C. The present inventors collected 0.5 to 0.6 mL of the supernatant, and transferred the supernatant to a new 1.5-mL microtube. The present inventors added isopropanol at 0.6 times the volume of the transferred solution, precipitated DNA, and then centrifuged the mixture in a refrigerated centrifuge at 15,000 rpm for five minutes at 4° C. The present inventors gently discarded the supernatant by pipetting, added 1 mL of 70% ethanol, and centrifuged the mixture again in a refrigerated centrifuge at 15,000 rpm for five minutes at 4° C. The present inventors discarded the supernatant, and dried the tube for approximately ten minutes with the tube unlidded. The precipitate was dissolved in 100 μL of TE buffer.

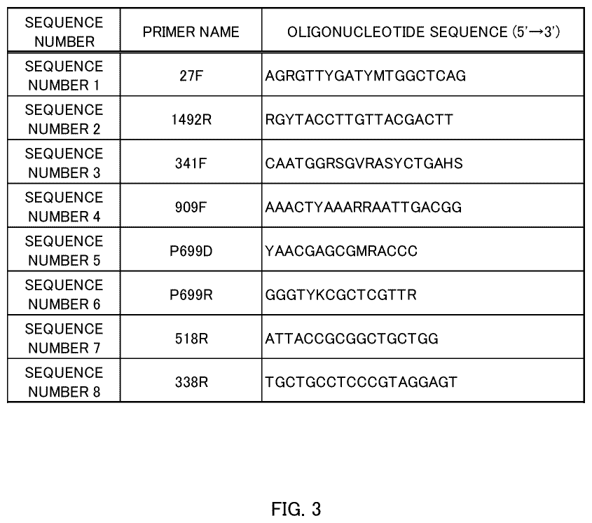

The present inventors prepared 19 μL of PCR reaction solution containing the 27F primer, the 1492R primer, TaKaRa LA Taq polymerase, and the like, and added, to the solution, 1 μL of DNA sample in which the precipitate was dissolved in a TE buffer. FIG. 3 depicts primers used in PCR reactions. FIG. 3 depicts the sequence numbers, primer names, and oligonucleotide sequences of the primers used in the PCR reactions. The first row from the top of FIG. 3 represents the oligonucleotide sequence of the 27F primer, and the second row from the top of FIG. 3 represents the oligonucleotide sequence of the 1492R primer. The present inventors spun down the tube using a simple centrifuge, inserted the tube into a constant-temperature block of a thermal cycler, and conducted 30 cycles of thermal cycle reaction.

### [Column Purification]

The present inventors added an equal amount of membrane binding solution to the DNA sample. The present inventors inserted an SV mini column into a recovery tube (also referred to as a column assembly). The present inventors transferred the entire solution to the SV mini column, and allowed the SV mini column to sit at room temperature for approximately one minute. The present inventors set the column assembly in a refrigerated centrifuge, and centrifuged the column assembly at 16,000×g for one minute at 4° C. The present inventors put 500 μL of membrane wash solution to the SV mini column, and centrifuged the mixture at 16,000×g for one minute at 4° C.

The present inventors discarded the liquid in the recovery tube into a waste reservoir, and inserted the SV mini column again into the recovery tube. The present inventors put 500 μL of membrane wash solution to the SV mini column, and centrifuged the mixture at 16,000×g for five minutes at 4° C. The present inventors discarded the liquid in the recovery tube into a waste reservoir, and centrifuged the column assembly having the SV mini column inserted into the recovery tube again at 16,000×g for one minute at 4° C. The present inventors inserted the SV mini column into a new 1.5-mL microtube. The present inventors put 50 μL of sterile water into the SV mini column, allowed the SV mini column to sit at room temperature for approximately one minute, then centrifuged the SV mini column at 16,000×g for one minute at 4° C., and then eluted the DNA.

### [Cycle Sequencing]

The present inventors prepared a sequencing reaction solution containing the 27F primer and the like, mixed 8 μL of the sequencing reaction solution and 2 μL of a sample, and conducted a reaction in a thermal cycler (initial denaturation: at 96° C. for one minute, [denaturation: at 96° C. for ten seconds, annealing: at 50° C. for five seconds, extension: at 60° C. for four seconds]×29 times, final extension: at 4° C. for an unrestricted duration). The present inventors used one of the primers with the sequence number 1 to the sequence number 8 depicted in FIG. 3 in the sequencing reaction solution. After the process in the thermal cycler, the present inventors added 5 μL of 125 mM EDTA and 60 μL of 99.5% EtOH, mixed the mixture by inversion, wrapped the mixture in aluminum foil, and allowed the mixture to sit for 15 minutes. Thereafter, the present inventors centrifuged the mixture at 3750×g for 30 minutes, and centrifuged the mixture at 185×g for ten seconds while the mixture is maintained in an inverted position.

The present inventors added 60 μL of 70% EtOH, centrifuged the mixture at 3750×g for five minutes, and further centrifuged the mixture at 185×g for ten seconds. The present inventors added 15 μL of HiDi formamide, vortexed the mixture for two minutes, subjected the mixture to heat shock at 95° C. for two minutes and at 4° C. for two minutes, and conducted capillary sequencing. The present inventors analyzed the DNA sequencing raw data by ATGC data analysis using ATGC software from GENETYX, and the analyzed data was subjected to BLAST analysis at NCBI. Then, the present inventors acquired FASTA data of closely-related strains using GENETYX software and the NCBI database, and constructed a phylogenetic tree of each isolate.

### [Selection of Microalgae Growth-Promoting Bacteria Through Co-Culture Evaluation System]

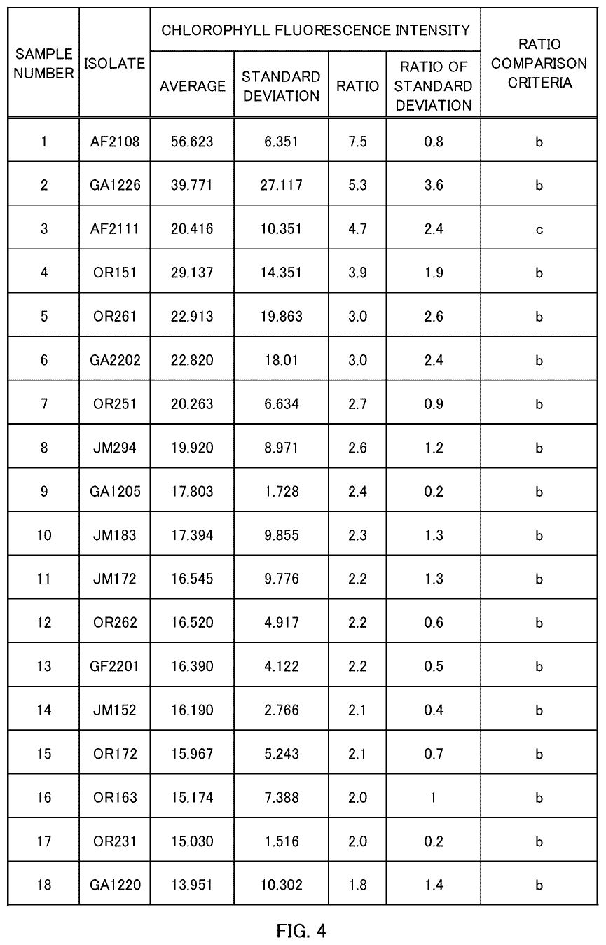

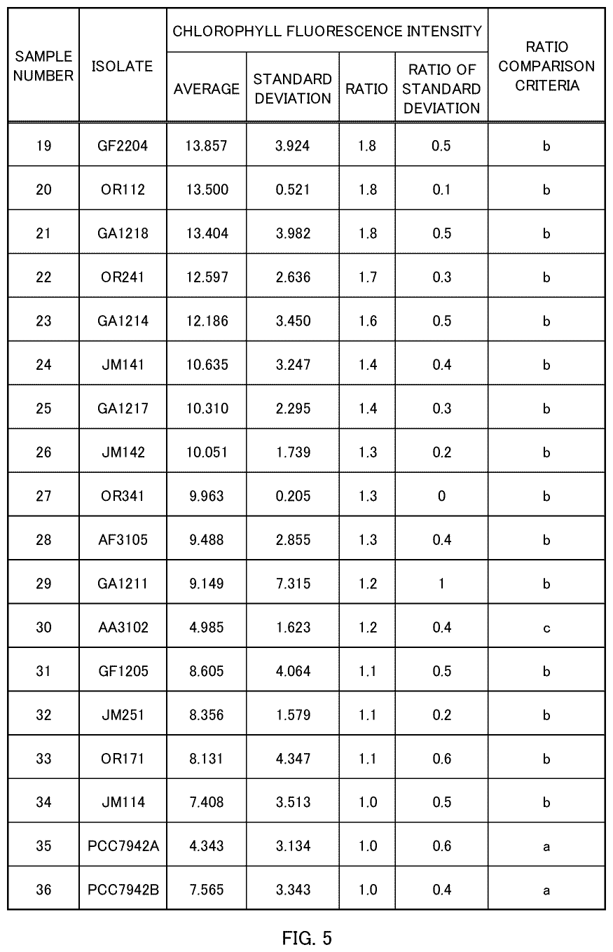

FIG. 4 and FIG. 5 depict isolates whose chlorophyll fluorescence intensity ratios on day 6 of co-culture exceeded 1.0. The fifth column from the left in FIG. 4 and FIG. 5 represents chlorophyll fluorescence intensity ratios compared with the case of culture with PCC7942 alone. The average, standard deviation, ratio, and ratio of standard deviation of the chlorophyll fluorescence intensity in the case of culture with PCC7942 alone are represented by the second row from the bottom in FIG. 5 (PCC7942A in FIG. 5) and the first row from the bottom in FIG. 5 (PCC7942B in FIG. 5).

The items marked with “a” in the ratio comparison criteria in the first column from the right in FIG. 4 and FIG. 5 represent the negative control. For the items marked with “b” in the ratio comparison criteria in the first column from the right in FIG. 4 and FIG. 5, the chlorophyll fluorescence intensity ratios compared with PCC7942A in FIG. 5 are represented by the fields for the ratios in the fifth column from the left in FIG. 4 and FIG. 5. For the items marked with “c” in the ratio comparison criteria in the first column from the right in FIG. 4 and FIG. 5, the chlorophyll fluorescence intensity ratios compared with PCC7942B in FIG. 5 are represented by the fields for the ratios in the fifth column from the left in FIG. 4 and FIG. 5.

Out of the 144 isolates from FCU, 34 strains have chlorophyll fluorescence intensity ratios which are equal to or greater than 1.0. FIG. 4 and FIG. 5 depict the 34 strains in descending order of the ratios. The isolate “AF2108” in the first row from the top in FIG. 4 exhibited the highest ratio of 7.5.

As depicted below, the present inventors selected isolates (AF2108 strain, GA1226 strain, AF2111 strain, OR151 strain) with chlorophyll fluorescence intensity ratios exceeding 3.0, and checked the PCC7942 growth-promoting effect and the NIES-2173 growth-promoting effect through co-culture in flasks.

### [AF2108 Isolate]

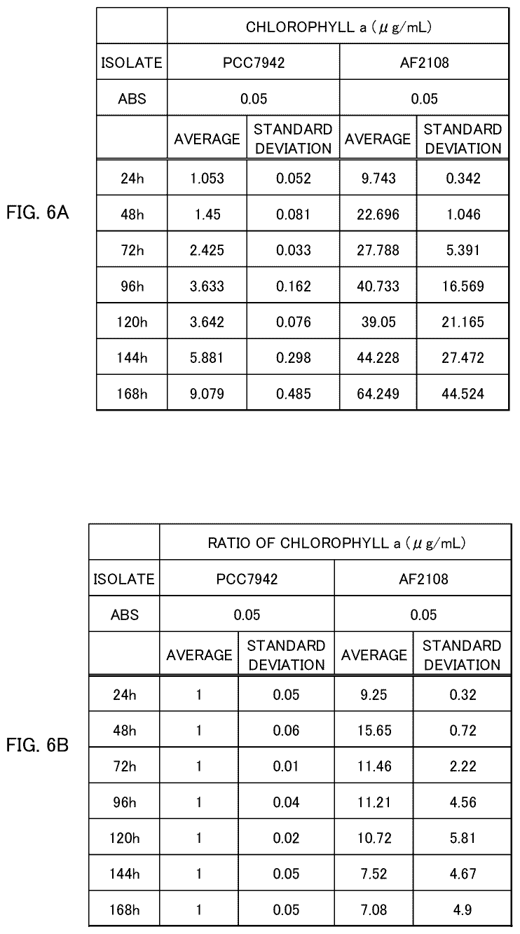

FIG. 6A and FIG. 6B depict the measurement results of chlorophyll a after the co-culture of the AF2108 isolate and PCC7942. The first column and second column from the left in FIG. 6A represent the average value and standard deviation of the chlorophyll a concentration during the monoculture of PCC7942, at 24-hour intervals. In the example of the first column and second column from the left in FIG. 6, the present inventors adjusted the amount of PCC7942 such that the absorbance at a wavelength of 730 nm at the start of the monoculture of PCC7942 became 0.05.

The third column and fourth column from the left in FIG. 6 represent the average value and standard deviation of the chlorophyll a concentration after the co-culture of PCC7942 and the AF2108 isolate, at 24-hour intervals. In the example of the third column and fourth column from the left in FIG. 6, the present inventors adjusted the amount of PCC7942 in the mixed co-culture medium of PCC7942 and the AF2108 isolate such that the absorbance at a wavelength of 730 nm of the PCC7942 component at the start of the co-culture became 0.05.

The present inventors adjusted the amount of the AF2108 isolate in the co-culture medium such that the absorbance at a wavelength of 600 nm of the AF2108 isolate component at the start of the co-culture became 0.05. After 168 hours of the monoculture of PCC7942, chlorophyll a increased to 9.079 μg/mL. On the other hand, after 168 hours of the co-culture of PCC7942 and the AF2108 isolate, chlorophyll a increased to 64.249 μg/mL.

FIG. 6B depicts the ratio obtained by dividing the chlorophyll a concentration during the co-culture of PCC7942 and the AF2108 isolate by the chlorophyll a concentration during the monoculture of PCC7942. As represented by the second row from the top in FIG. 6B, the ratio comparing the chlorophyll a concentration during the co-culture of PCC7942 and the AF2108 isolate with the monoculture of PCC7942 became a maximum of 15.65±0.72 at 48 hours, and became a minimum of 7.08±4.90 at 168 hours.

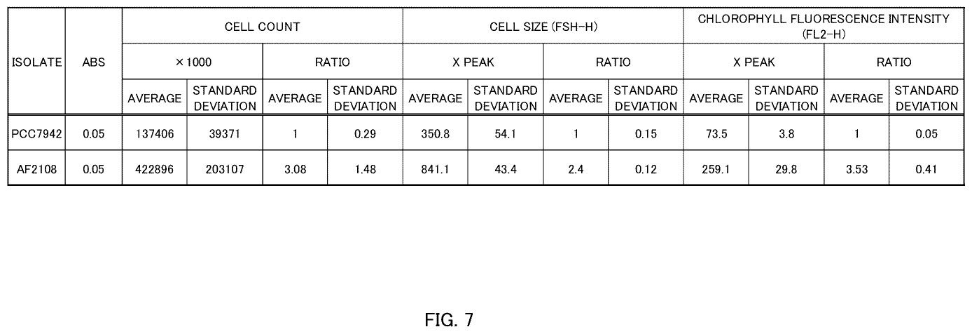

FIG. 7 depicts the results of flow cytometry measurements of the cell count, cell size, and chlorophyll fluorescence intensity of PCC7942. The first row from the top in FIG. 7 represents the cell count and the like of PCC7942 after the monoculture of PCC7942 was continued for 168 hours. The second row from the top in FIG. 7 represents the cell count and the like of PCC7942 after the co-culture of PCC7942 and the AF2108 isolate was continued for 168 hours. The present inventors measured the cell size using forward scatter (FSC-H) in flow cytometry. The cell size and chlorophyll fluorescence intensity after 168 hours of the co-culture of PCC7942 and the AF2108 isolate became 2.40±0.12 times and 3.53±0.41 times, respectively, compared with the monoculture of PCC7942.

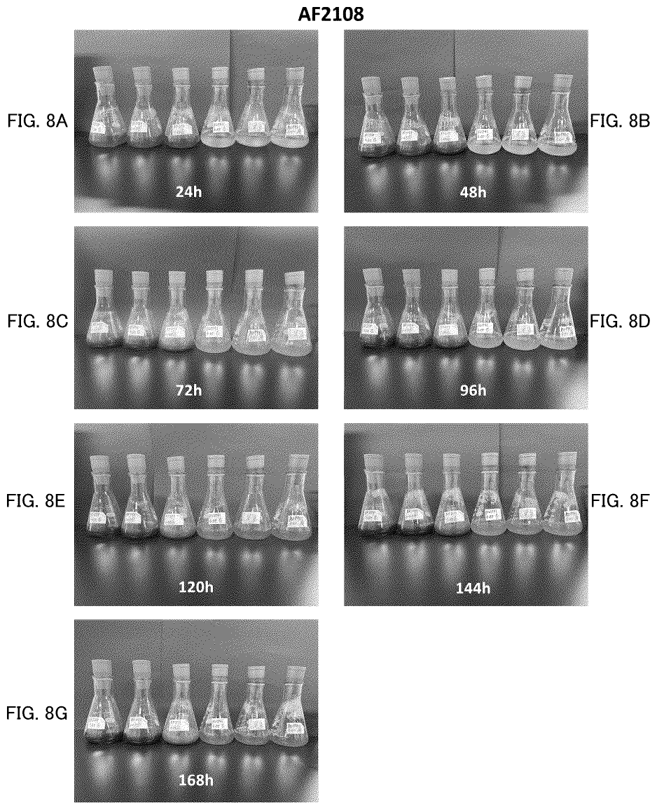

FIG. 8A to FIG. 8G depict, in chronological order, the state of the co-culture of PCC7942 and the AF2108 isolate in flasks. It was confirmed that samples during the co-culture of PCC7942 and the AF2108 isolate after 24 hours (the first to third ones from the left) were far greener than samples during the monoculture of PCC7942 (the fourth to sixth ones from the left).

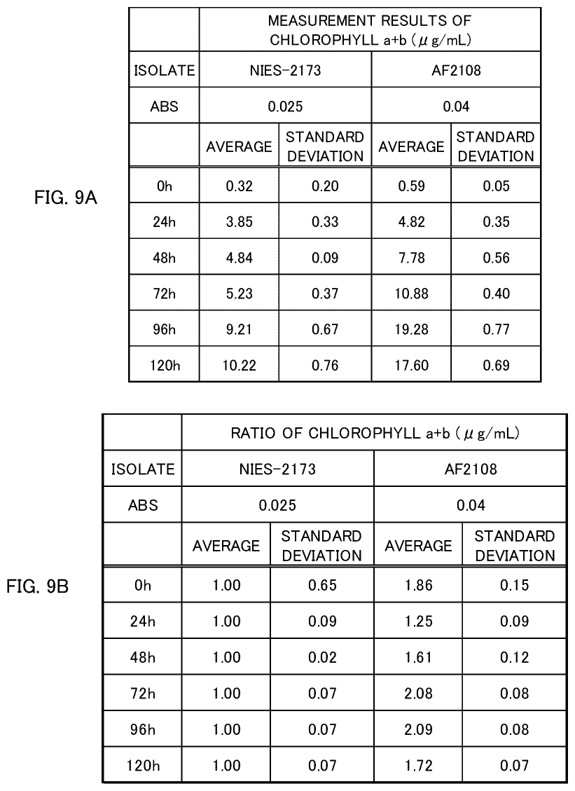

FIG. 9A and FIG. 9B depict the measurement results of the total of the chlorophyll a and chlorophyll b concentrations during the co-culture of the AF2108 isolate and NIES-2173 (hereinafter also referred to as chlorophyll a+b concentration). The first column and second column from the left in FIG. 9A represent the average value and standard deviation of the chlorophyll a+b concentration during the monoculture of Chlorella NIES-2173, at 24-hour intervals. In the example of the first column and second column from the left in FIG. 9, the present inventors adjusted the amount of NIES-2173 such that the absorbance at a wavelength of 750 nm at the start of the monoculture of NIES-2173 became 0.025.

The third column and fourth column from the left in FIG. 9 represent the average value and standard deviation of the chlorophyll a+b concentration during the co-culture of NIES-2173 and the AF2108 isolate, at 24-hour intervals. In the example of the third column and fourth column from the left in FIG. 9, the present inventors adjusted the amount of AF2108 in the mixed co-culture medium ofNIES-2173 and the AF2108 isolate such that the absorbance at a wavelength of 600 nm of the AF2108 component at the start of the co-culture became 0.04.

After 120 hours of the monoculture of NIES-2173, the chlorophyll a+b concentration increased to 10.22 μg/mL. On the other hand, after 120 hours of the co-culture of NIES-2173 and the AF2108 isolate, the chlorophyll a+b concentration increased to 17.60 μg/mL.

FIG. 9B depicts the ratio obtained by dividing the chlorophyll a+b concentration during the co-culture of NIES-2173 and the AF2108 isolate by the chlorophyll a+b concentration during the monoculture of NIES-2173. As depicted in FIG. 9(b), the ratio comparing the chlorophyll a+b concentration during the co-culture of NIES-2173 and the AF2108 isolate with the chlorophyll a+b concentration during the monoculture of NIES-2173 became a maximum of 2.09±0.08 at 96 hours, and became a minimum of 1.25±0.09 at 24 hours. Accordingly, the present inventors confirmed that the AF2108 isolate promotes the growth of NIES-2173.

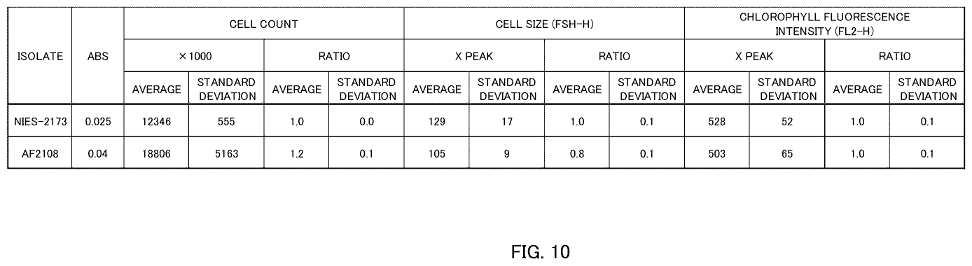

FIG. 10 depicts the results of flow cytometry measurements of the cell count, cell size, and chlorophyll fluorescence intensity of NIES-2173. The first row from the top in FIG. 10 represents the cell count and the like of NIES-2173 after the monoculture of NIES-2173. The second row from the top in FIG. 10 represents the cell count and the like of NIES-2173 after the co-culture of NIES-2173 and the AF2108 isolate. The present inventors measured the cell size using forward scatter (FSC-H) in flow cytometry. The cell size and chlorophyll fluorescence intensity after the co-culture of NIES-2173 and the AF2108 isolate became 0.8±0.1 times and 1.0±0.1 times, respectively, compared with the monoculture of NIES-2173.

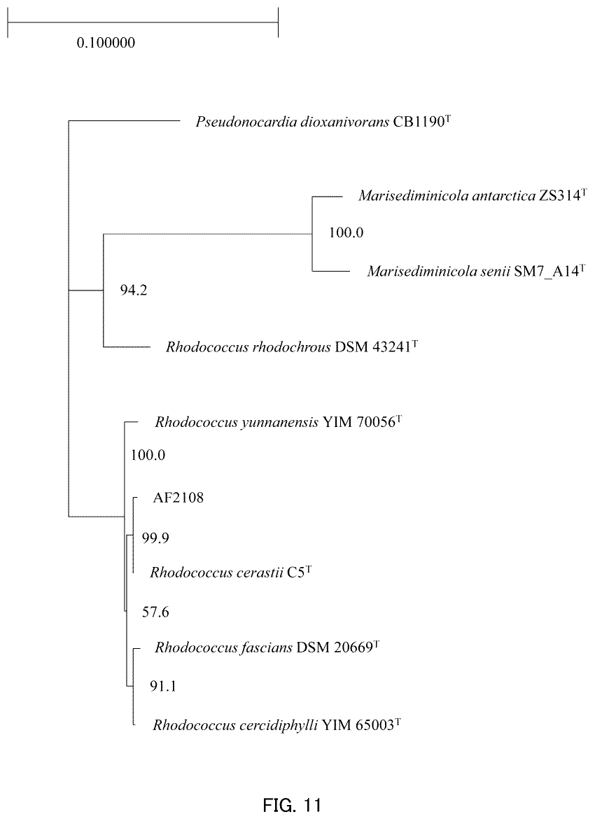

FIG. 11 depicts the phylogenetic tree of Rhodococcus. The Pseudonocardia dioxanivorans CP1190 standard strain was treated as an outgroup. The numerical values in FIG. 11 are bootstrap values representing the reliability of forms in the phylogenetic tree. Only bootstrap values which are equal to or greater than 50 are depicted. The scale bar represents the scale of the number of amino acid substitutions. From the phylogenetic tree, the AF2108 strain is most closely related to Rhodococcus cerastii (Rhodococcus cerastii). The AF2108 strain matches Rhodococcus cerastii with 100% homology, and was identified as Rhodococcus cerastii (deposit number: NITE P-03678).

### [AF2111 Isolate]

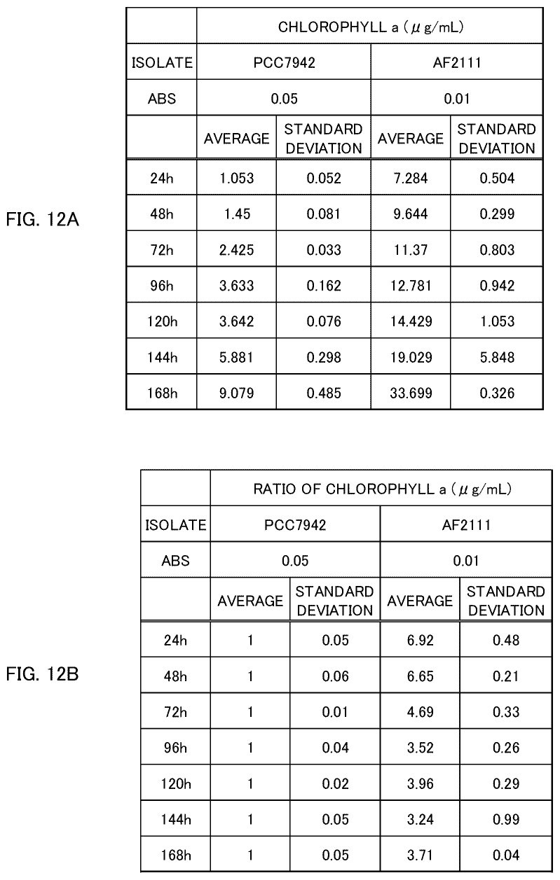

FIG. 12A and FIG. 12B depict the measurement results of chlorophyll a during the co-culture of PCC7942 and the AF2111 isolate. The first column and second column from the left in FIG. 12A represent the average value and standard deviation of the chlorophyll a concentration during the monoculture of PCC7942, at 24-hour intervals. In the example of the monoculture of PCC7942 depicted in FIG. 12, the present inventors adjusted the amount of PCC7942 such that the absorbance at a wavelength of 730 nm at the start of the monoculture of PCC7942 became 0.05.

The third column and fourth column from the left in FIG. 12 represent the average value and standard deviation of the chlorophyll a concentration during the co-culture of PCC7942 and the AF2111 isolate, at 24-hour intervals. In the example of the co-culture of PCC7942 and the AF2111 isolate in FIG. 12, the present inventors adjusted the amount of PCC7942 in the mixed co-culture medium of PCC7942 and the AF2111 isolate such that the absorbance at a wavelength of 730 nm of the PCC7942 component at the start of the co-culture became 0.05, and adjusted the amount of the AF2111 isolate in the mixed co-culture medium of PCC7942 and the AF2111 isolate such that the absorbance at a wavelength of 600 nm of the AF2111 isolate component at the start of the co-culture became 0.01.

As represented by the first row from the bottom in FIG. 12A, in the monoculture of PCC7942, chlorophyll a increased to 9.079 μg/mL at 168 hours of culture. On the other hand, in the co-culture of PCC7942 and the AF2111 isolate, chlorophyll a increased to 33.699 μg/mL at 168 hours of culture.

FIG. 12B depicts the ratio obtained by dividing the chlorophyll a concentration during the co-culture of PCC7942 and the AF2111 isolate by the chlorophyll a concentration during the monoculture of PCC7942. This ratio became a maximum of 6.92±0.48 at 24 hours, and became a minimum of 3.24±0.99 at 144 hours.

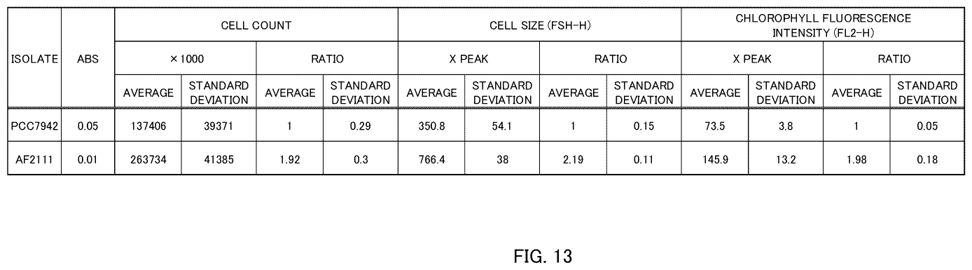

FIG. 13 depicts the results of flow cytometry measurements of the cell count, cell size, and chlorophyll fluorescence intensity of PCC7942. The first row from the top in FIG. 13 represents the cell count and the like of PCC7942 after the monoculture of PCC7942 was continued for 168 hours. The second row from the top in FIG. 13 represents the cell count and the like of PCC7942 after the co-culture of PCC7942 and the AF2111 isolate was continued for 168 hours. As represented by the second row from the top in FIG. 13, the cell size and chlorophyll fluorescence intensity after the co-culture of PCC7942 and the AF2111 isolate became 2.19±0.11 times and 1.98±0.18 times, respectively, compared with the cell size and the like after the monoculture of PCC7942.

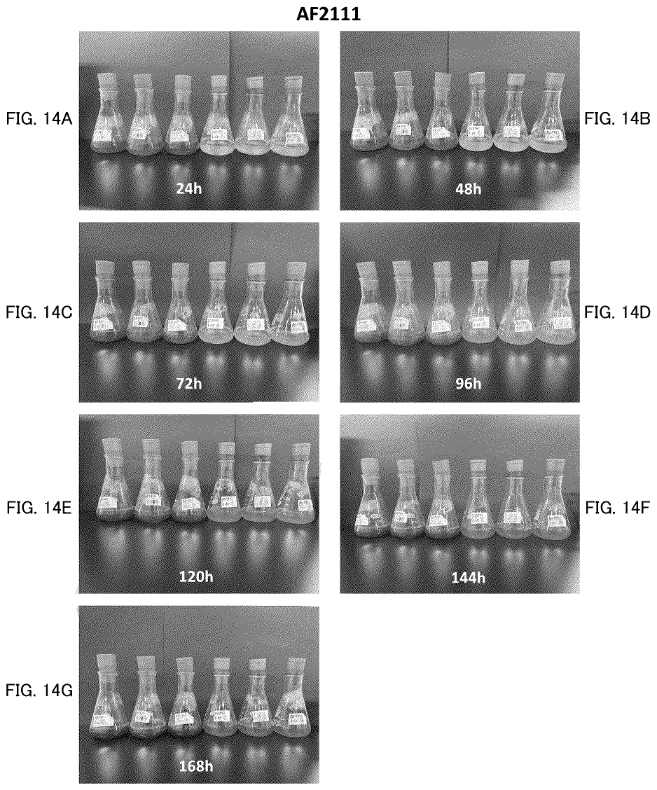

FIG. 14A to FIG. 14G depict, in chronological order, the state of the co-culture of PCC7942 and the AF2111 isolate in flasks. It was confirmed that samples during the co-culture of PCC7942 and the AF2111 isolate after 24 hours (the first to third ones from the left) were far greener than samples during the monoculture of PCC7942 (the fourth to sixth ones from the left).

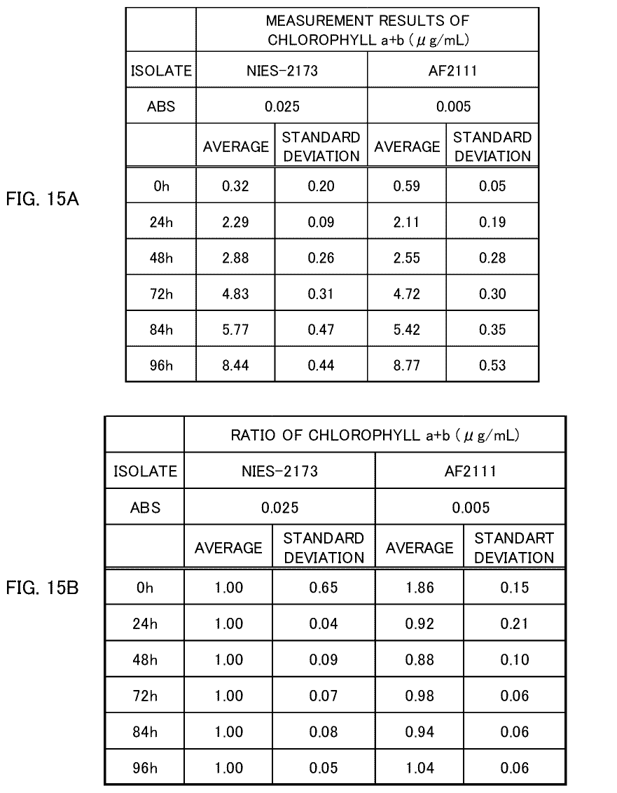

FIG. 15A and FIG. 15B depict the measurement results of the chlorophyll a+b concentration during the co-culture of the AF2111 isolate and NIES-2173. The first column and second column from the left in FIG. 15A represent the average value and standard deviation of the chlorophyll a+b concentration during the monoculture of Chlorella NIES-2173, at 24-hour intervals, and also represent the average value and the like of the chlorophyll a+b concentration after 84 hours. In the example of the first column and second column from the left in FIG. 15, the present inventors adjusted the amount of NIES-2173 such that the absorbance at a wavelength of 750 nm at the start of the monoculture of NIES-2173 became 0.025.

The third column and fourth column from the left in FIG. 15 represent the average value and standard deviation of the chlorophyll a+b concentration during the co-culture of NIES-2173 and the AF2111 isolate. In the example of the third column and fourth column from the left in FIG. 15, the present inventors adjusted the amount of AF2111 in the mixed co-culture medium of NIES-2173 and the AF2111 isolate such that the absorbance at a wavelength of 600 nm of the AF2111 component at the start of the co-culture became 0.005.

After 96 hours of the monoculture of NIES-2173, the chlorophyll a+b concentration increased to 8.44 μg/mL. On the other hand, after 96 hours of the co-culture of NIES-2173 and the AF2111 isolate, the chlorophyll a+b concentration increased to 8.77 μg/mL.

FIG. 15B depicts the ratio obtained by dividing the chlorophyll a+b concentration during the co-culture of NIES-2173 and the AF2111 isolate by the chlorophyll a+b concentration during the monoculture of NIES-2173. As depicted in FIG. 15B, the ratio comparing the chlorophyll a+b concentration during the co-culture of NIES-2173 and the AF2111 isolate with the chlorophyll a+b concentration during the monoculture of NIES-2173 became a maximum of 1.04±0.06 at 96 hours, and became a minimum of 0.88±0.10 at 48 hours, excluding 1.86±0.15 at 0 hours.

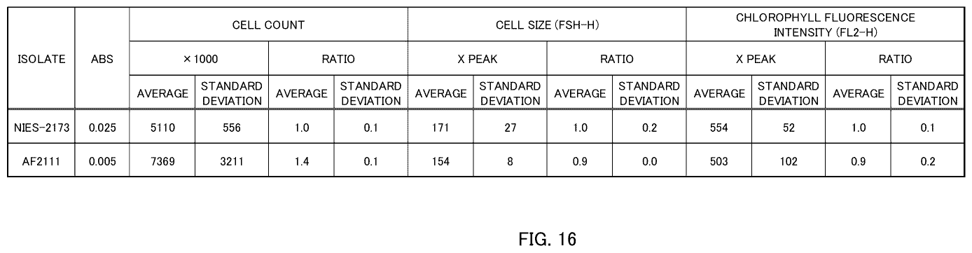

FIG. 16 depicts the results of flow cytometry measurements of the cell count, cell size, and chlorophyll fluorescence intensity of NIES-2173. The first row from the top in FIG. 16 represents the cell count and the like of NIES-2173 after the monoculture of NIES-2173. The second row from the top in FIG. 16 represents the cell count and the like of NIES-2173 after the co-culture of NIES-2173 and the AF2111 isolate. The present inventors measured the cell size using forward scatter (FSC-H) in flow cytometry.

The cell size and chlorophyll fluorescence intensity after the co-culture of NIES-2173 and the AF2111 isolate became 0.9±0.0 times and 0.9±0.2 times, respectively, compared with the monoculture of NIES-2173.

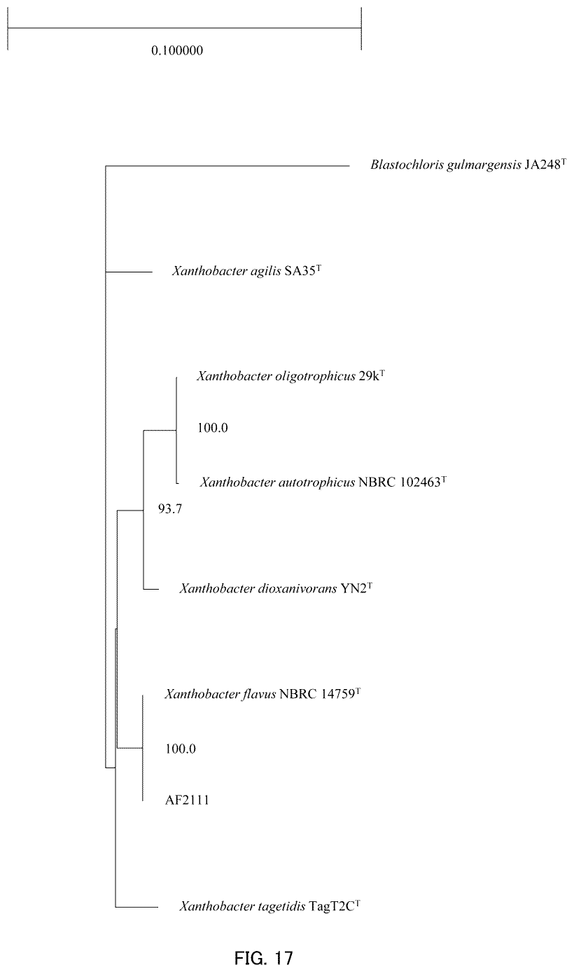

FIG. 17 depicts the phylogenetic tree of Xanthobacter. The present inventors treated the Blastochloris gulmargensis JA248 type strain as an outgroup. From this phylogenetic tree, the AF2111 strain is most closely related to Xanthobacter flavus (Xanthobacter flavus). The AF2111 strain matches Xanthobacter flavus with 100% homology, and was identified as Xanthobacter flavus (deposit number: NITE P-03679). Accordingly, it was demonstrated that Xanthobacter flavus has a microalgae growth-promoting effect.

### [GA1226 Isolate]

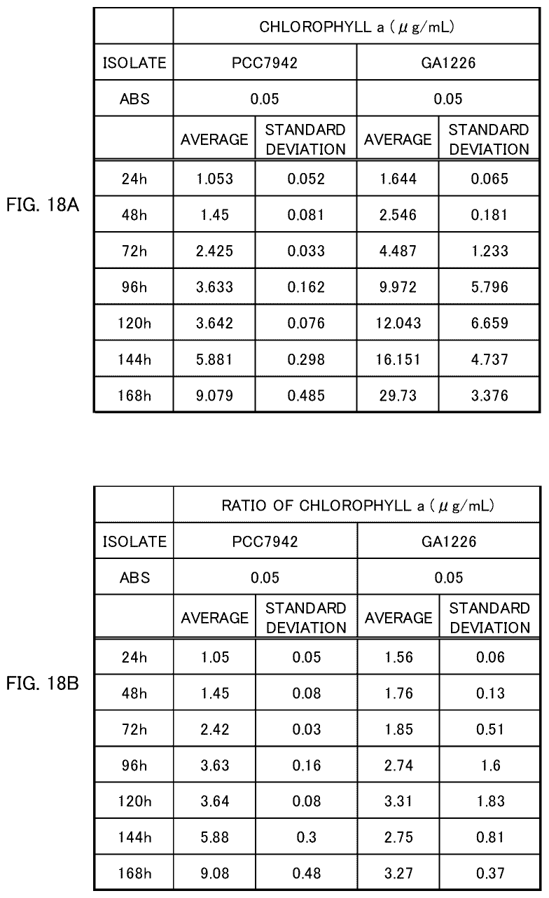

FIG. 18A and FIG. 18B depict the measurement results of chlorophyll a during the co-culture of PCC7942 and the GA1226 isolate. The first column and second column from the left in FIG. 18A represent the average value and standard deviation of the chlorophyll a concentration during the monoculture of PCC7942, at 24-hour intervals. The present inventors adjusted the monoculture of PCC7942 such that the absorbance at a wavelength of 730 nm at the start of the culture of PCC7942 became 0.05.

The third column and fourth column from the left in FIG. 18 represent the average value and standard deviation of the chlorophyll a concentration during the co-culture of PCC7942 and the GA1226 isolate, at 24-hour intervals. The present inventors adjusted the amount of PCC7942 in the co-culture of PCC7942 and the GA1226 isolate such that the absorbance at a wavelength of 730 nm at the start of the co-culture of the PCC7942 component became 0.05 in the mixed co-culture medium of PCC7942 and the GA1226 isolate. The present inventors adjusted the amount of the GA1226 isolate in the co-culture medium such that the absorbance at a wavelength of 600 nm at the start of the co-culture of the GA1226 isolate component became 0.05. As represented by the first row from the bottom in FIG. 18A, in the monoculture of PCC7942, chlorophyll a increased to 9.079 μg/mL after 168 hours of culture. On the other hand, in the co-culture of PCC7942 and the GA1226 isolate, chlorophyll a increased to 29.73 μg/mL after 168 hours of culture.

FIG. 18B depicts the ratio obtained by dividing the chlorophyll a concentration after the co-culture of PCC7942 and the GA1226 isolate by the chlorophyll a concentration after the monoculture of PCC7942. This ratio became a maximum of 3.31±1.83 (120 hours), and, even at its lowest, became 1.56±0.06 (24 hours).

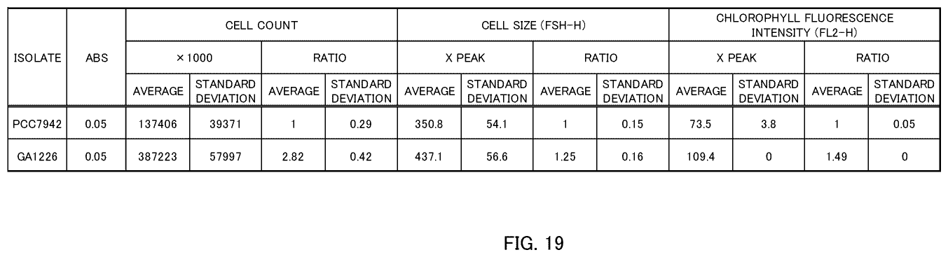

FIG. 19 depicts the results of flow cytometry measurements of the cell count, cell size, and chlorophyll fluorescence intensity of PCC7942. The first row from the top in FIG. 19 represents the cell count and the like of PCC7942 after the monoculture of PCC7942 was continued for 168 hours. The second row from the top in FIG. 19 represents the cell count and the like of PCC7942 after the co-culture of PCC7942 and the GA1226 isolate was continued for 168 hours. The cell size and chlorophyll fluorescence intensity after the co-culture of PCC7942 and the GA1226 isolate became 1.25±0.16 times and 1.49±0 times, respectively, compared with the cell size and the like after the monoculture of PCC7942.

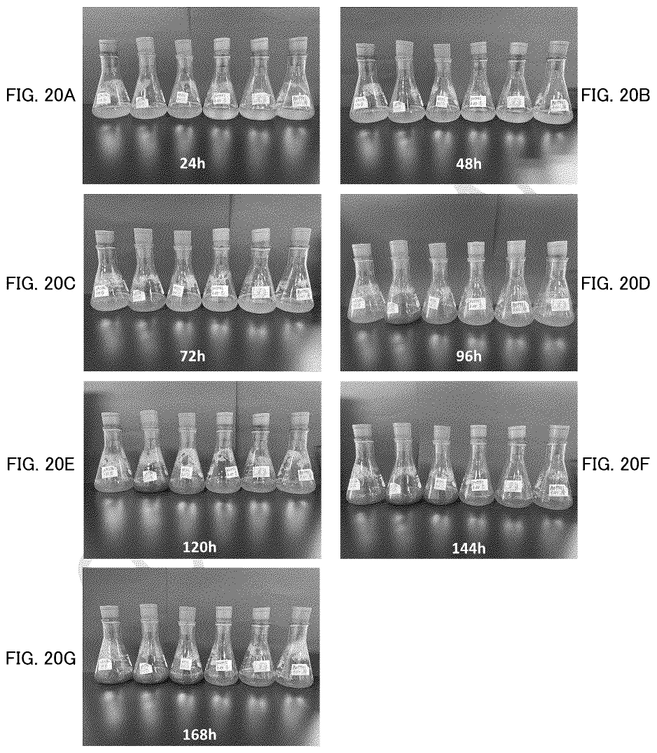

FIG. 20A to FIG. 20G depict, in chronological order, the state of the co-culture of PCC7942 and the GA1226 isolate in flasks. It was confirmed that samples during the co-culture of PCC7942 and the GA1226 isolate after 48 hours (the first to third ones from the left) were far greener than samples during the monoculture of PCC7942 (the fourth to sixth ones from the left).

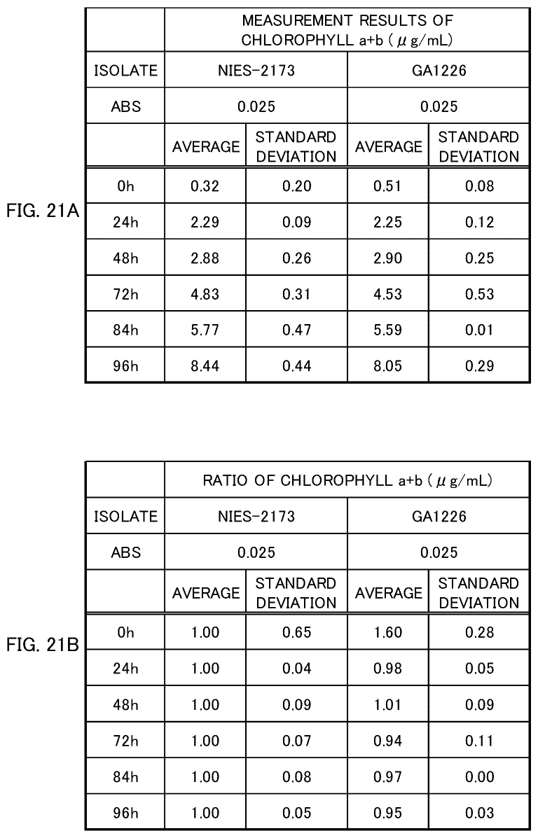

FIG. 21A and FIG. 21B depict the measurement results of the chlorophyll a+b concentration during the co-culture of the GA1226 isolate and NIES-2173. The first column and second column from the left in FIG. 21A represent the average value and standard deviation of the chlorophyll a+b concentration during the monoculture of Chlorella NIES-2173, at 24-hour intervals, and also represent the average value and the like of the chlorophyll a+b concentration after 84 hours. In the example of the first column and second column from the left in FIG. 21, the present inventors adjusted the amount of NIES-2173 such that the absorbance at a wavelength of 750 nm at the start of the monoculture of NIES-2173 became 0.025.

The third column and fourth column from the left in FIG. 21 represent the average value and standard deviation of the chlorophyll a+b concentration during the co-culture of NIES-2173 and the GA1226 isolate. In the example of the third column and fourth column from the left in FIG. 21, the present inventors adjusted the amount of GA1226 in the mixed co-culture medium of NIES-2173 and the GA1226 isolate such that the absorbance at a wavelength of 600 nm of the GA1226 component at the start of the co-culture became 0.025. After 96 hours of the monoculture of NIES-2173, the chlorophyll a+b concentration increased to 8.44 μg/mL. On the other hand, after 96 hours of the co-culture of NIES-2173 and the GA1226 isolate, the chlorophyll a+b concentration increased to 8.05 μg/mL.

FIG. 21B depicts the ratio obtained by dividing the chlorophyll a+b concentration during the co-culture of NIES-2173 and the GA1226 isolate by the chlorophyll a+b concentration during the monoculture of NIES-2173. As depicted in FIG. 21B, the ratio comparing the chlorophyll a+b concentration during the co-culture of NIES-2173 and the GA1226 isolate with the chlorophyll a+b concentration during the monoculture of NIES-2173 became a maximum of 0.97±0.00 at 84 hours, and became a minimum of 0.94±0.11 at 72 hours, excluding 1.60±0.28 at 0 hours.

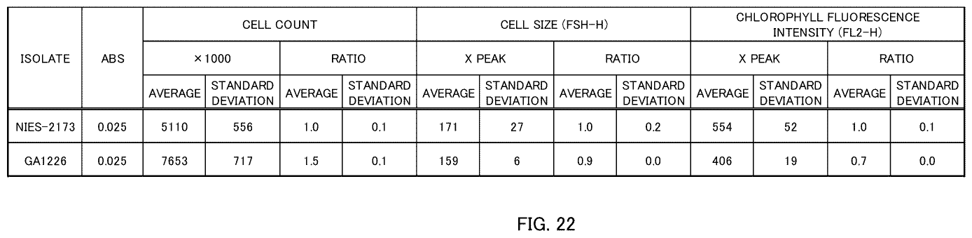

FIG. 22 depicts the results of flow cytometry measurements of the cell count, cell size, and chlorophyll fluorescence intensity of NIES-2173. The first row from the top in FIG. 22 represents the cell count and the like of NIES-2173 after the monoculture of NIES-2173. The second row from the top in FIG. 21 represents the cell count and the like of NIES-2173 after the co-culture of NIES-2173 and the GA1226 isolate. The present inventors measured the cell size using forward scatter (FSC-H) in flow cytometry. The cell size and chlorophyll fluorescence intensity after the co-culture of NIES-2173 and the GA1226 isolate became 0.9±0.0 times and 0.7±0.0 times, respectively, compared with the monoculture of NIES-2173.

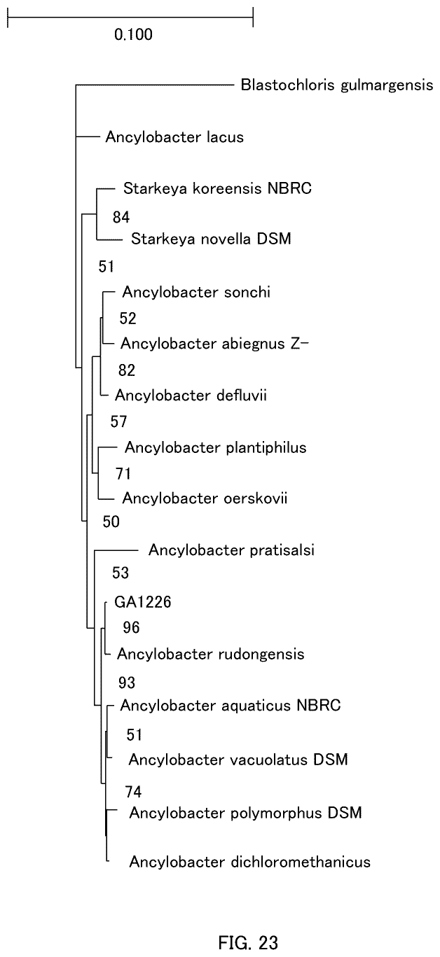

FIG. 23 depicts the phylogenetic tree of Ancylobacter. The present inventors treated the Blastochloris gulmargensis JA248T type strain as an outgroup. From the phylogenetic tree, the GA1226 isolate is most closely related to Ancylobacter rudongensis (Ancylobacter rudongensis). The GA1226 isolate matches Ancylobacter rudongensis with 99.42% homology, and was identified as Ancylobacter rudongensis (deposit number: NITE P-03779). Accordingly, it was demonstrated that Ancylobacter rudongensis has a microalgae growth-promoting effect.

### [OR151 Isolate]

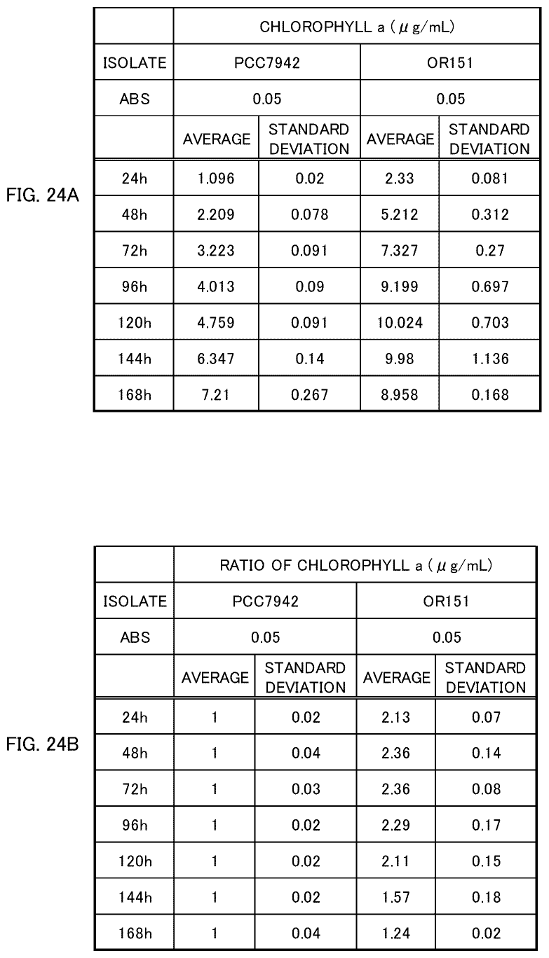

FIG. 24A and FIG. 24B depict the measurement results of chlorophyll a during the co-culture of PCC7942 and the OR151 isolate. The first column and second column from the left in FIG. 24A represent the average value and standard deviation of the chlorophyll a concentration during the monoculture of PCC7942, at 24-hour intervals. The present inventors adjusted the amount of PCC7942 in the monoculture of PCC7942 such that the absorbance at a wavelength of 730 nm at the start of the culture of PCC7942 became 0.05.

The third column and fourth column from the left in FIG. 24 represent the average value and standard deviation of the chlorophyll a concentration during the co-culture of PCC7942 and the OR151 isolate, at 24-hour intervals. The present inventors adjusted the amount of PCC7942 in the co-culture of PCC7942 and the OR151 isolate such that the absorbance at a wavelength of 730 nm at the start of the culture of the PCC7942 component became 0.05 in the mixed co-culture medium of PCC7942 and the OR151 isolate. The present inventors adjusted the amount of the OR151 isolate in the co-culture medium such that the absorbance at a wavelength of 600 nm at the start of the culture of the OR151 isolate component became 0.05. The present inventors confirmed that while, as represented by the first row from the bottom in FIG. 24A, chlorophyll a increased to 7.21 μg/mL after 168 hours of culture in the monoculture of PCC7942, chlorophyll a increased to 8.958 μg/mL after 168 hours of culture in the co-culture of PCC7942 and the OR151 isolate.

FIG. 24B depicts the ratio obtained by dividing the chlorophyll a concentration during the co-culture of PCC7942 and the OR151 isolate by the chlorophyll a concentration during the monoculture of PCC7942. This ratio became a maximum of 2.36±0.14 (48 hours) and 2.36±0.08 (72 hours), and became a minimum of 1.24±0.02 (168 hours).

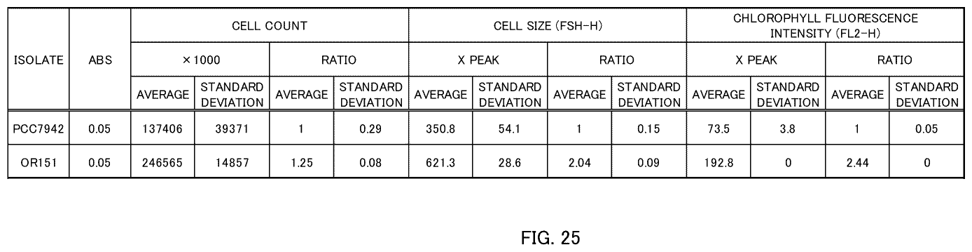

FIG. 25 depicts the results of flow cytometry measurements of the cell count, cell size, and chlorophyll fluorescence intensity of PCC7942. The first row from the top in FIG. 25 represents the cell count and the like of PCC7942 after the monoculture of PCC7942 was continued for 168 hours. The second row from the top in FIG. 25 represents the cell count and the like of PCC7942 after the co-culture of PCC7942 and the OR151 isolate was continued for 168 hours. The cell size and chlorophyll fluorescence intensity after 168 hours of the co-culture of PCC7942 and the OR151 isolate became 2.04±0.09 times and 2.44±0 times, respectively, compared with the cell size and the like after the monoculture of PCC7942.

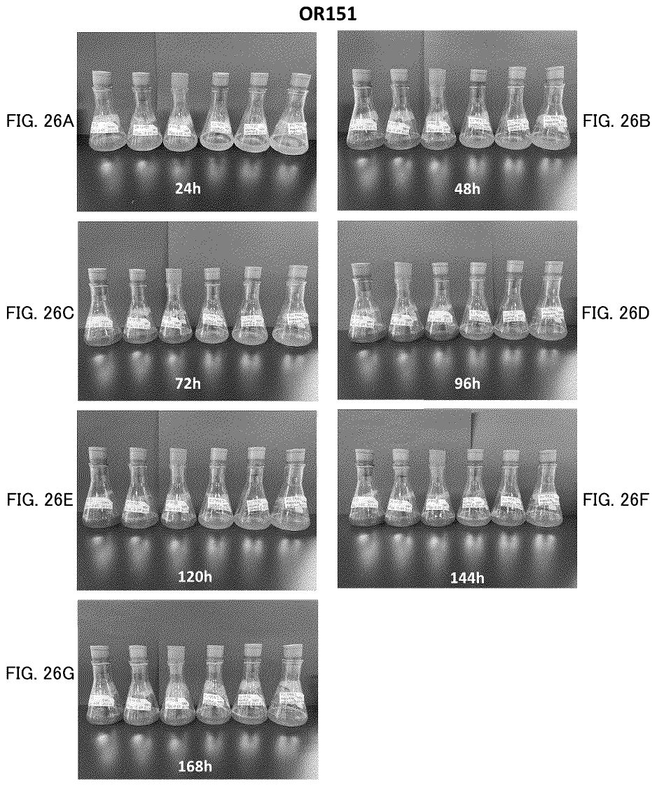

FIG. 26A to FIG. 26G depict, in chronological order, the state of the co-culture of PCC7942 and the OR151 isolate in flasks. It was confirmed that samples during the co-culture of PCC7942 and the OR151 isolate after 24 hours (the first to third ones from the left) were far greener than samples during the monoculture of PCC7942 (the fourth to sixth ones from the left).

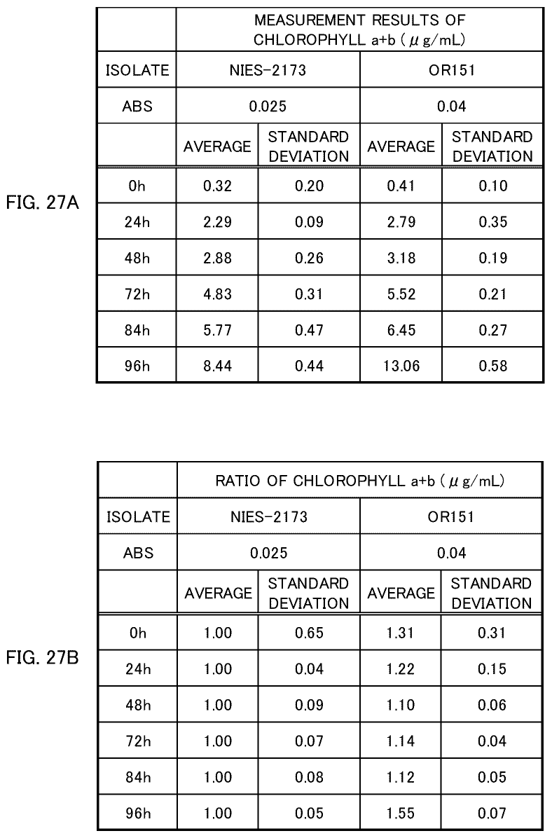

FIG. 27A and FIG. 27B depict the measurement results of the chlorophyll a+b concentration during the co-culture of the OR151 isolate and NIES-2173. The first column and second column from the left in FIG. 27A represent the average value and standard deviation of the chlorophyll a+b concentration during the monoculture of Chlorella NIES-2173, at 24-hour intervals, and also represent the average value and the like of the chlorophyll a+b concentration after 84 hours. In the example of the first column and second column from the left in FIG. 27, the present inventors adjusted the amount of NIES-2173 such that the absorbance at a wavelength of 750 nm at the start of the monoculture of NIES-2173 became 0.025.

The third column and fourth column from the left in FIG. 27 represent the average value and standard deviation of the chlorophyll a+b concentration during the co-culture of NIES-2173 and the OR151 isolate. In the example of the third column and fourth column from the left in FIG. 21, the present inventors adjusted the amount of OR151 in the mixed co-culture medium of NIES-2173 and the OR151 isolate such that the absorbance at a wavelength of 600 nm of the OR151 component at the start of the co-culture became 0.04. After 96 hours of the monoculture of NIES-2173, the chlorophyll a+b concentration increased to 8.44 μg/mL. On the other hand, after 96 hours of the co-culture of NIES-2173 and the OR151 isolate, the chlorophyll a+b concentration increased to 13.06 μg/mL.

FIG. 27B depicts the ratio obtained by dividing the chlorophyll a+b concentration during the co-culture of NIES-2173 and the OR151 isolate by the chlorophyll a+b concentration during the monoculture of NIES-2173. As depicted in FIG. 27(b), the ratio comparing the chlorophyll a+b concentration during the co-culture of NIES-2173 and the OR151 isolate with the monoculture of NIES-2173 became a maximum of 1.55±0.07 at 96 hours, and became a minimum of 1.10±0.06 at 48 hours, excluding 1.31±0.31 at 0 hours. Accordingly, it was demonstrated that the OR151 isolate has an NIES-2173 growth-promoting effect.

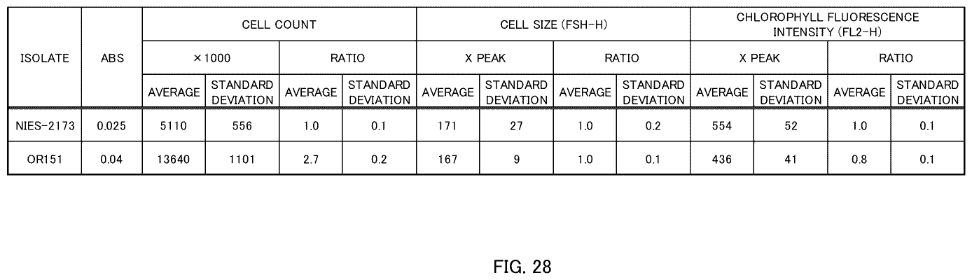

FIG. 28 depicts the results of flow cytometry measurements of the cell count, cell size, and chlorophyll fluorescence intensity of NIES-2173. The first row from the top in FIG. 28 represents the cell count and the like of NIES-2173 after the monoculture of NIES-2173. The second row from the top in FIG. 28 represents the cell count and the like of NIES-2173 after the co-culture of NIES-2173 and the OR151 isolate. The present inventors measured the cell size using forward scatter (FSC-H) in flow cytometry. The cell size and chlorophyll fluorescence intensity after the co-culture of NIES-2173 and the OR151 isolate became 1.0±0.1 times and 0.8±0.1 times, respectively, compared with the monoculture of NIES-2173.

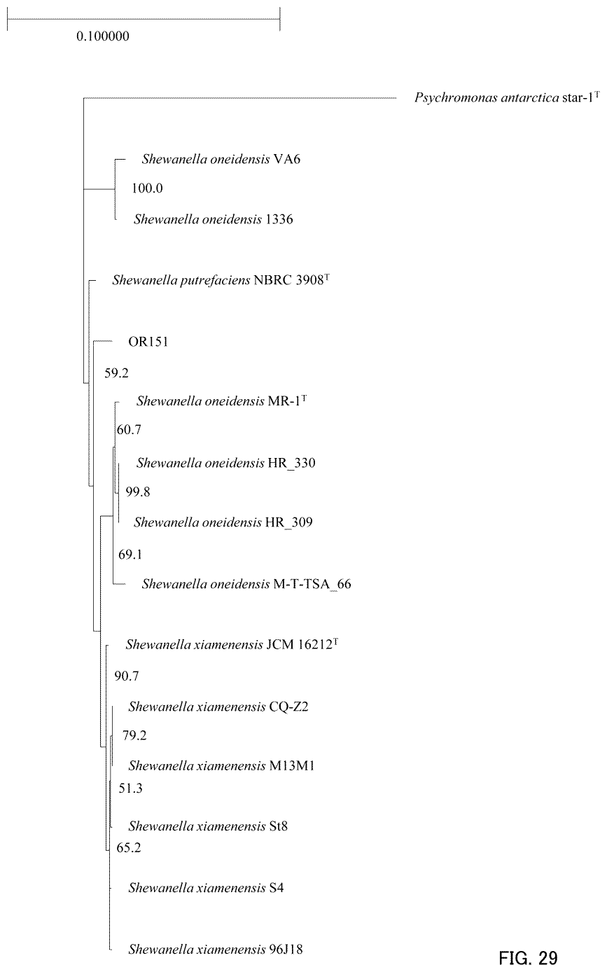

FIG. 29 depicts the phylogenetic tree of Shewanella. The present inventors treated the Psychromonas antarctica star-1 type strain as an outgroup. From this phylogenetic tree, the OR151 isolate relatively closely related to the species Shewanella oneidensis (Shewanella oneidensis). However, the homology between the OR151 isolate and Shewanella oneidensis is lower than the homology between the OR151 isolate and Shewanella putrefaciens (Shewanella putrefaciens) and the homology between the OR151 isolate and Shewanella profunda (Shewanella profunda). It has been known that the OR151 isolate is a microorganism classified into Shewanella sp., but its species name has not been identified (deposit number: NITE P-03682). It was demonstrated that the OR151 isolate, accordingly microorganisms of Shewanella sp., has a microalgae growth-promoting effect.

### [Microalgae Growth-Promoting Microorganism Isolation and Culture Methods]

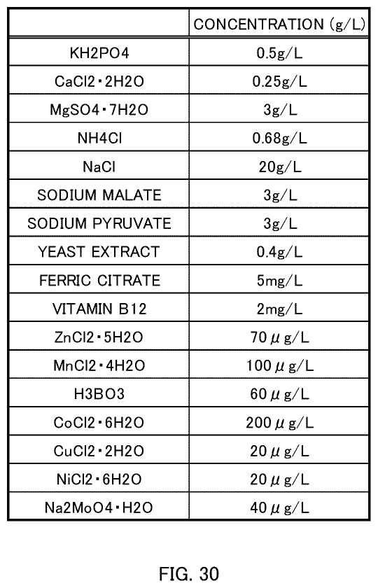

The present inventors isolated microorganisms having the microalgae growth-promoting effect using a JCM520 medium. The components of the JCM520 medium are depicted in FIG. 30. The present inventors added 2% agar in addition to each medium component depicted in FIG. 30, and implemented autoclaving at 121° C. for 20 minutes to prepare a JCM520 agar medium. The present inventors used FCU having been fermented as the isolation source, and prepared a liquid in which the isolation-source FCU was diluted in the range of ten times to 10⁵ times. The present inventors sowed 100 μL of the diluted FCU onto the solidified JCM520 medium in each petri dish, inoculated the diluted FCU using a Conradi rod, put lids on the petri dishes, and cultured microorganisms in an environment under aerobic conditions, at room temperature (24° C.), with 115 to 120 μmol/m²/s light exposure for 12 hours out of every 24 hours until colonies were formed. The present inventors used a disposable loop to pick up the formed colonies, subcultured the formed colonies several times onto a medium similar to the medium used for the isolation, and obtained a single colony.

### [Summary of Screening of Microalgae Growth-Promoting Bacteria by High-Throughput Co-Culture Evaluation System]

### (1) Euglena Algae

The present inventors used Euglena gracilis NIES-48 (hereinafter also referred to as “NIES-48” simply), which is a model organism of Euglena algae, in order to evaluate the microalgae growth-promoting effect. In the case of the monoculture of NIES-48, the present inventors used a modified CM medium as a preculture to perform flask culture at 25° C. and 100 rpm with a photon flux density of 150 μmol/m²/s under continuous light exposure for 24 hours for seven days. The photon flux density represents the intensity of light.

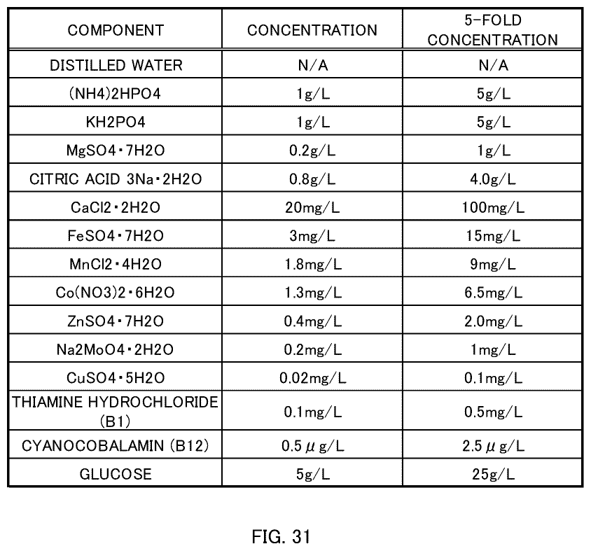

The present inventors inoculated NIES-48 in the modified CM medium (the composition is depicted in FIG. 31) without mixing NIES-48 with isolates. The composition of the modified CM medium is depicted in FIG. 31. After inoculating NIES-48 onto the modified CM medium, the present inventors performed main culture at 25° C. and 100 rpm with a photon flux density of 150 μmol/m²/s under continuous light exposure for 24 hours for seven days. The present inventors measured the chlorophyll a concentration (μg/mL) and the chlorophyll b concentration (μg/mL) at 24-hour intervals.

In a case where isolates isolated from FCU and NIES-48 are co-cultured, as a preculture for the co-culture, the present inventors used a 100-mL flask, inoculated 2 mL of strain glycerol stock solution in 40 mL of modified CM medium, and cultured microorganisms at 30° C. and 168 rpm for two days. During the main culture, the present inventors dispensed 40 mL of co-culture medium into a 100-mL flask, adjusted the amount of NIES-48 such that the light absorbance A730 at a wavelength of 730 nm of NIES-48 became 0.05 after mixing with isolates, and adjusted the amount of each isolate isolated from FCU such that the light absorbance A600 at a wavelength of 600 nm of the isolate became 0.01, 0.05, or the like after mixing with NIES-48.

The present inventors inoculated NIES-48 onto a modified CM medium after mixing NIES-48 and isolates, and performed main culture at 25° C. and 100 rpm with a photon flux density of 150 μmol/m²/s under continuous light exposure for 24 hours for seven days. The present inventors quantified the chlorophyll a concentration (mg/L) and the chlorophyll b concentration (mg/L) at 24-hour intervals. The present inventors confirmed that each isolate did not exhibit chlorophyll fluorescence similar to the chlorophyll fluorescence of NIES-48. This demonstrates that the respective isolates do not affect the measurement results of the chlorophyll fluorescence intensity derived from NIES-48 during the co-culture with NIES-48.

### <Chlorophyll Quantification>

The present inventors recovered 1 mL of sample after the co-culture, and centrifuged the sample at 4° C. and 6,000×g for ten minutes. The present inventors removed the supernatant fraction, and resuspended cells, recovered as a pellet, in an 80% (vol/vol) acetone aqueous solution. The present inventors allowed the mixture to sit at 4° C. for one hour, then centrifuged the mixture at 4° C. and 6,000×g for ten minutes, and recovered the supernatant in a 1.5-mL tube to obtain a pigment extract. In order to prevent condensation on the cuvette surface, the present inventors returned the extract to room temperature, then transferred the extract to a glass cuvette, and measured absorbance at 646 nm, 663 nm, and 750 nm. Note that, in order to calibrate the effects of turbidity and colored compounds, the present inventors set 750 nm as the zero point on the spectrophotometer.

The present inventors calculated each pigment content on the basis of the following computation formulae. ChlA represents chlorophyll A, and ChlB represents chlorophyll B.

$$ChlA ( mg / L ) = 12.21 ( Abs 663 - Abs 750 ) - 2.81 ( Abs 646 - Abs 750 )$$

$$ChlB ( mg / L ) = 20.13 ( Abs 646 - Abs 750 ) - 5.03 ( Abs 663 - Abs 750 )$$

When measuring the absorbance at each wavelength, the present inventors performed the measurement after measuring the blank using an 80% (v/v) acetone aqueous solution.

### <Flow Cytometry Analysis>

The present inventors measured the cell count and the chlorophyll fluorescence per cell using the flow cytometer Cube8. The present inventors prepared 1 mL of each culture solution diluted 20 times, and treated them as measurement samples. The present inventors set the voltages for forward scatter (FSC-H), side scatter (SSC-H), and chlorophyll fluorescence (FL2-H) to 125.0 V, 180.0 V, 525.0 V, and 350.0 V, respectively.

### [Genome Extraction of Microalgae Growth-Promoting Strain]

The present inventors conducted monoculture of each isolate, and subjected it to genome extraction. The present inventors centrifuged a tube containing bacterial cells in a refrigerated centrifuge at 10,000 rpm and 4° C. for five minutes, and recovered a cell pellet (precipitate). The present inventors discarded the supernatant into a waste reservoir using a pipette. The present inventors put 560 μL of TE buffer into the cell pellet, thoroughly agitated the mixture, and suspended the cells. The present inventors added 30 μL of 10% SDS and 10 μL of proteinase K solution, thoroughly mixed the mixture, and then kept the mixture warm at 37° C. for one hour. The present inventors added 100 μL of 5M NaCl, and thoroughly mixed the mixture. The present inventors added 80 μL of CTAB/NaCl solution, thoroughly mixed the mixture, and kept the mixture warm at 65° C. for ten minutes. The present inventors put in 0.7 mL of chloroform/isoamyl alcohol, closed the lid of the tube, inverted the tube upside down five to six times, thoroughly agitated the mixture, and then centrifuged the mixture in a refrigerated centrifuge at 15,000 rpm for five minutes at 4° C.

The present inventors collected 0.5 to 0.6 mL of the upper layer of the liquid, and transferred the collected liquid to a new 1.5-mL microtube. The present inventors added an equal amount of phenol/chloroform/isoamyl alcohol to the transferred liquid amount, thoroughly agitated the mixture, and then centrifuged the mixture in a refrigerated centrifuge at 15,000 rpm for five minutes at 4° C. The present inventors collected 0.5 to 0.6 mL of the supernatant, and transferred the supernatant to a new 1.5-mL microtube. The present inventors added isopropanol at 0.6 times the volume of the transferred solution, precipitated DNA, and then centrifuged the mixture in a refrigerated centrifuge at 15,000 rpm for five minutes at 4° C. The present inventors gently discarded the supernatant by pipetting, added 1 mL of 70% ethanol, and centrifuged the mixture again in a refrigerated centrifuge at 15,000 rpm for five minutes at 4° C. The present inventors discarded the supernatant, and dried the tube for approximately ten minutes with the tube unlidded. The present inventors dissolved the precipitate in 100 μL of TE buffer.

The present inventors prepared 19 μL of PCR reaction solution containing the 27F primer, the 1492R primer, TaKaRa LA Taq polymerase, and the like, and added, to the solution, 1 μL of DNA sample in which the precipitate was dissolved in a TE buffer. FIG. 3 depicts primers used in PCR reactions. FIG. 3 depicts the sequence numbers, primer names, and oligonucleotide sequences of the primers used in the PCR reactions. The first row from the top of FIG. 3 represents the oligonucleotide sequence of the 27F primer (sequence number 1), and the second row from the top of FIG. 3 represents the oligonucleotide sequence of the 1492R primer (sequence number 2). The present inventors spun down the tube using a simple centrifuge, inserted the tube into a constant-temperature block of a thermal cycler, and conducted 30 cycles of thermal cycle reaction.

### [Column Purification]

The present inventors added an equal amount of membrane binding solution to the DNA sample. The present inventors inserted an SV mini column into a recovery tube (also referred to as a column assembly). The present inventors transferred the entire solution to the SV mini column, and allowed the SV mini column to sit at room temperature for approximately one minute. The present inventors set the column assembly in a refrigerated centrifuge, and centrifuged the column assembly at 16,000×g for one minute at 4° C. The present inventors put 500 μL of membrane wash solution to the SV mini column, and centrifuged the mixture at 16,000×g for one minute at 4° C.

The present inventors discarded the liquid in the recovery tube into a waste reservoir, and inserted the SV mini column again into the recovery tube. The present inventors put 500 μL of membrane wash solution to the SV mini column, and centrifuged the mixture at 16,000×g for five minutes at 4° C. The present inventors discarded the liquid in the recovery tube into a waste reservoir, and centrifuged the column assembly having the SV mini column inserted into the recovery tube again at 16,000×g for one minute at 4° C. The present inventors inserted the SV mini column into a new 1.5-mL microtube. The present inventors put 50 μL of sterile water into the SV mini column, allowed the SV mini column to sit at room temperature for approximately one minute, then centrifuged the SV mini column at 16,000×g for one minute at 4° C., and then eluted the DNA.

### [Cycle Sequencing]

The present inventors prepared a sequencing reaction solution containing the 27F primer and the like, mixed 8 μL of the sequencing reaction solution and 2 μL of a sample, and conducted a reaction in a thermal cycler (initial denaturation: at 96° C. for one minute, [denaturation: at 96° C. for ten seconds, annealing: at 50° C. for five seconds, extension: at 60° C. for four seconds]×29 times, final extension: at 4° C. for an unrestricted duration). The present inventors used one of the primers with the sequence number 1 to the sequence number 8 depicted in FIG. 3 in the sequencing reaction solution. After the process in the thermal cycler, the present inventors added 5 μL of 125 mM EDTA and 60 μL of 99.5% EtOH, mixed the mixture by inversion, wrapped the mixture in aluminum foil, and allowed the mixture to sit for 15 minutes. Thereafter, the present inventors centrifuged the mixture at 3750×g for 30 minutes, and centrifuged the mixture at 185×g for ten seconds while the mixture is maintained in an inverted position.

The present inventors added 60 μL of 70% EtOH, centrifuged the mixture at 3750×g for five minutes, and further centrifuged the mixture at 185×g for ten seconds. The present inventors added 15 μL of HiDi formamide, vortexed the mixture for two minutes, subjected the mixture to heat shock at 95° C. for two minutes and at 4° C. for two minutes, and conducted capillary sequencing. The present inventors analyzed the DNA sequencing raw data by ATGC data analysis using ATGC software from GENETYX, and the analyzed data was subjected to BLAST analysis at NCBI. Then, the present inventors acquired FASTA data of closely-related strains using GENETYX software and the NCBI database, and constructed a phylogenetic tree of each isolate.

### [Selection of Microalgae Growth-Promoting Bacteria Through Co-Culture Evaluation System]

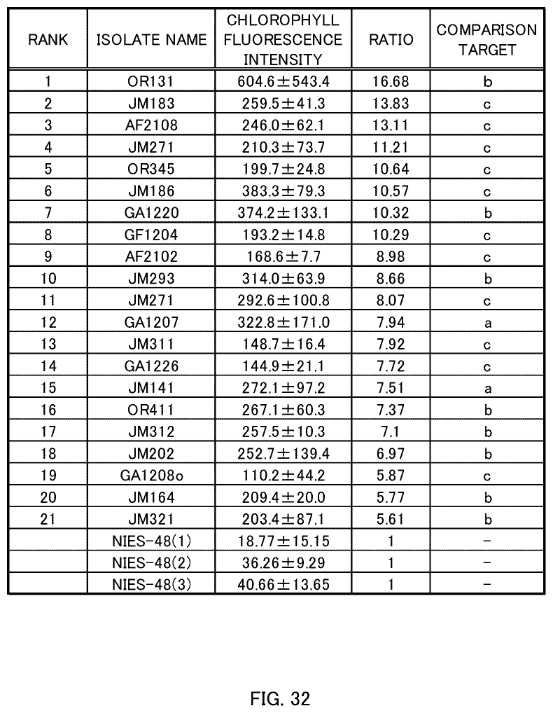

FIG. 32 depicts isolates whose chlorophyll fluorescence intensity ratios at 168 hours of co-culture became equal to or greater than 5.6 out of those whose chlorophyll fluorescence intensity ratios exceeded 1.0, in descending order of increase ratio. The isolate names were given for convenience by the present inventors and the like. The “ratio” in FIG. 32 represents chlorophyll fluorescence intensity ratios compared with the case of the culture with NIES-48 alone.

The chlorophyll fluorescence intensity depicted in FIG. 32 represents the average value of the results of multiple measurements of the chlorophyll fluorescence intensity. Items marked with “a” in the comparison target fields represent ratios compared with NIES-48(1) represented by the third row from the bottom in FIG. 32. Items marked with “b” in the comparison target fields represent ratios compared with NIES-48(2) represented by the second row from the bottom in FIG. 32. Items marked with “c” in the comparison target fields represent ratios compared with NIES-48(3) represented by the first row from the bottom in FIG. 32.

Out of the 144 isolates from FCU, 21 strains have chlorophyll fluorescence intensity ratios which are equal to or greater than 5.6.

In the following case, the present inventors selected the JM311 strain, the JM321 strain, the AF2108 strain, and the JM202 strain from the isolates whose chlorophyll fluorescence intensity ratios exceeded 5.6, and checked the NIES-48 growth-promoting effect through co-culture in flasks.

### [JM311 Isolate]

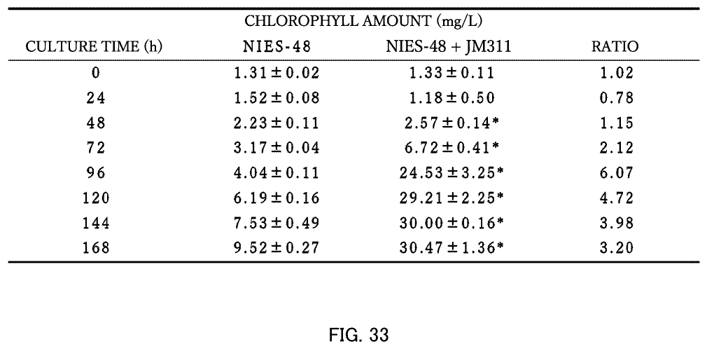

FIG. 33 depicts the measurement results of chlorophyll a+b after the co-culture of the JM311 isolate and NIES-48. Marks * in FIG. 33 represent that there is a significant difference from the monoculture of NIES-48 (p<0.05). The co-culture of NIES-48 and the JM311 strain made the chlorophyll amount at 168 hours of culture 30.47 mg/L, and increased the chlorophyll amount to 3.20 times compared to 9.52 mg/L during the monoculture.

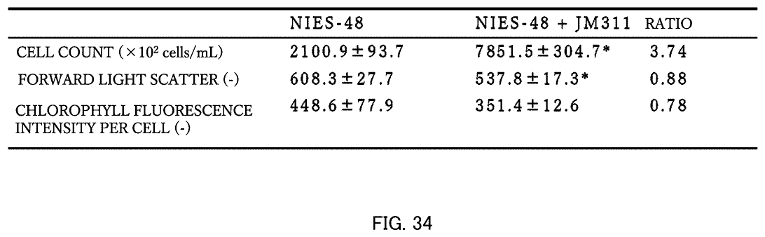

FIG. 34 depicts the results of flow cytometry measurements of the cell count, cell size, and chlorophyll fluorescence intensity of NIES-48 during the monoculture of NIES-48 or during the co-culture with JM311. The present inventors measured the cell size using forward scatter (FSC-H) in flow cytometry. Marks * in FIG. 34 represent that there is a significant difference from the monoculture of NIES-48 (p<0.05). The cell count after 168 hours of the co-culture of NIES-48 and the JM311 isolate increased to 3.74 times compared with the monoculture of NIES-48. The chlorophyll fluorescence intensity per cell remained unchanged at 0.78 times. The forward light scatter slightly decreased to 0.88 times. This revealed that the co-culture of NIES-48 and JM311 promotes the growth, and increases the cell count.

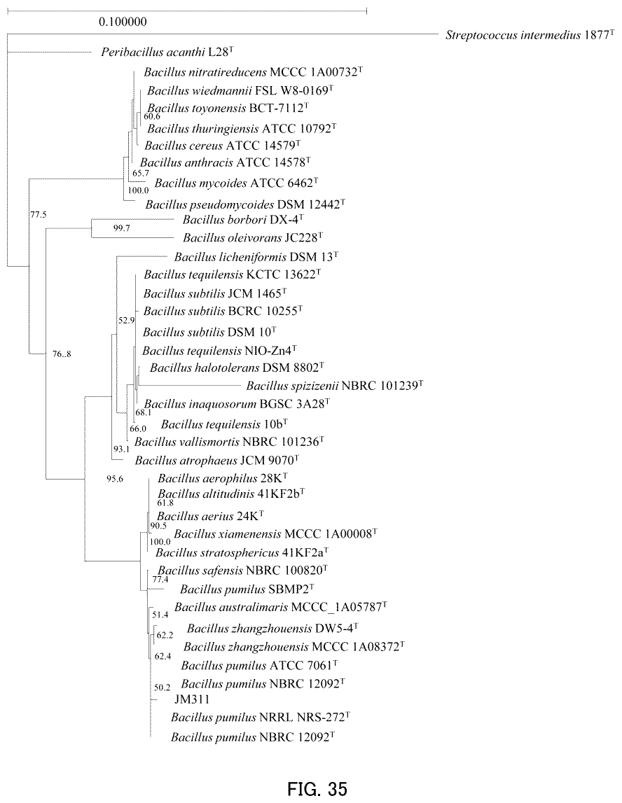

The present inventors determined the almost complete 16S rRNA sequence of the JM311 strain through sequencing (1383 bp). According to the results of a sequence analysis using BLASTn, the top five strains with high homology were Bacillus pumilus ATCC7061, Bacillus pumilus NBRC12092, Bacillus zhangzhouensis MCCC1A08372, Peribacillus acanthi L28, and Bacillus australimaris MCCC1A05787, with homology of 99.86%, 99.86%, 99.78%, 99.78%, and 99.71%, respectively. FIG. 35 depicts the phylogenetic tree of Bacillus closely related to JM311. The present inventors constructed the phylogenetic tree depicted in FIG. 35 on the basis of the 16S rRNA sequence of JM311. Bootstrap values which are equal to or greater than 50 are depicted. The present inventors used the Streptococcus intermedius 1877 type strain as an outgroup. JM311 has been deposited in National Institute of Technology and Evaluation, Patent Microorganisms Depositary Center (deposit number: NITE P-03919).

### [JM321 Isolate]

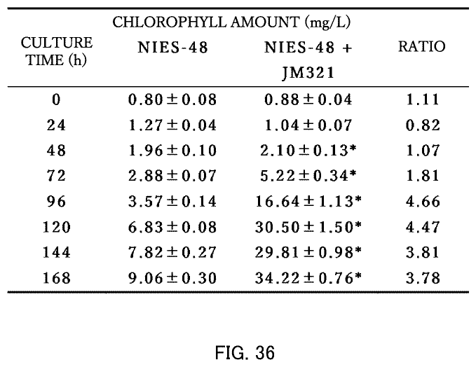

FIG. 36 depicts the measurement results of chlorophyll a+b after the co-culture of the JM321 isolate and NIES-48. Marks * in FIG. 36 represent that there is a significant difference from the monoculture of NIES-48 (p<0.05). The co-culture of NIES-48 and the JM321 strain made the chlorophyll amount at 168 hours of culture 34.22 mg/L, and increased the chlorophyll amount to 3.78 times compared to 9.06 mg/L during the monoculture.

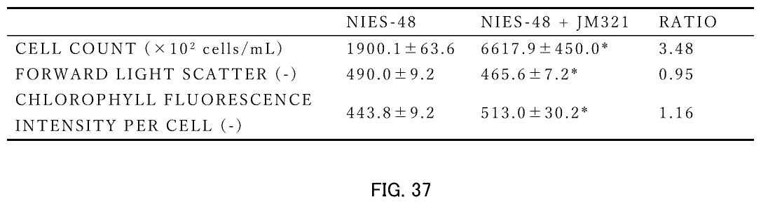

FIG. 37 depicts the results of flow cytometry measurements of the cell count, cell size, and chlorophyll fluorescence intensity of NIES-48 during the monoculture of NIES-48 or during the co-culture with JM321. The present inventors measured the cell size using forward scatter (FSC-H) in flow cytometry. Marks * in FIG. 37 represent that there is a significant difference from the monoculture of NIES-48 (p<0.05). The cell count and chlorophyll fluorescence intensity after 168 hours of the co-culture of NIES-48 and the JM321 isolate were 3.48 times and 1.16 times, respectively, compared with the monoculture of NIES-48. The forward light scatter became 0.95 times. This revealed that the co-culture of NIES-48 and JM321 slightly increases the chlorophyll amount per cell, promotes the growth, and increases the cell count.

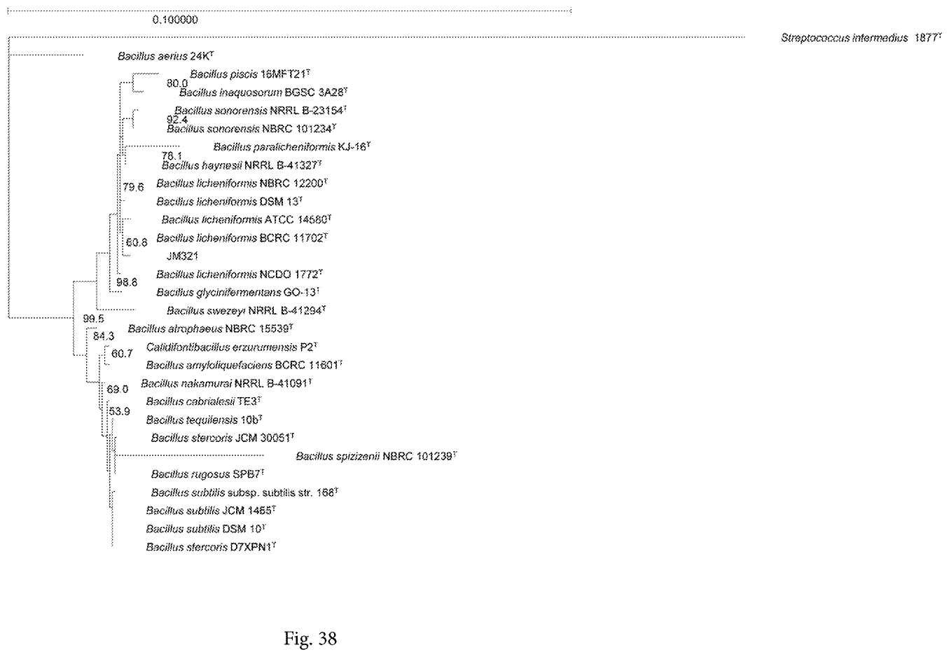

The present inventors determined the almost complete 16S rRNA sequence of the JM321 strain through sequencing (1483 bp). According to the results of a sequence analysis using BLASTn, the top five strains with high homology were Bacillus licheniformis DSM13, Bacillus licheniformis BCRC11702, Bacillus paralicheniformis KJ-16, Bacillus licheniformis NRRL B-41327, and Bacillus licheniformis ATCC14580, with homology of 99.85%, 99.85%, 99.76%, 99.69%, and 99.691%, respectively. FIG. 38 depicts the phylogenetic tree of Bacillus closely related to JM321. The present inventors constructed the phylogenetic tree depicted in FIG. 38 on the basis of the 16S rRNA gene sequence of JM321. Bootstrap values which are equal to or greater than 50 are depicted. The present inventors used the Streptococcus intermedius 1877 type strain as an outgroup. The present inventors identified JM321 as Bacillus licheniformis. JM321 has been deposited in National Institute of Technology and Evaluation, Patent Microorganisms Depositary Center (deposit number: NITE P-03920).

### [AF2108 Isolate]

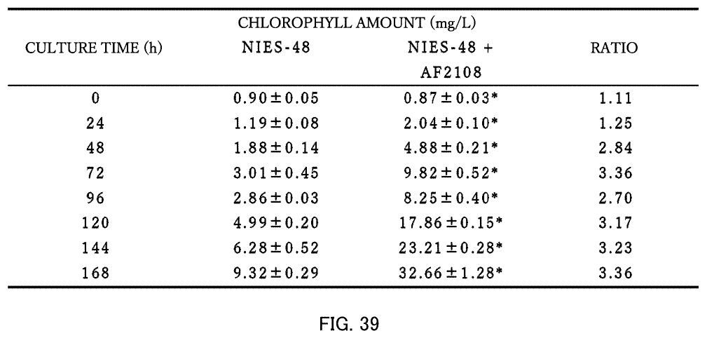

FIG. 39 depicts the measurement results of chlorophyll a+b after the co-culture of the AF2108 isolate and NIES-48. Marks * in FIG. 39 represent that there is a significant difference from the monoculture of NIES-48 (p<0.05). The co-culture of NIES-48 and the AF2108 strain made the chlorophyll amount at 168 hours of culture 32.66 mg/L, and increased the chlorophyll amount to 3.36 times compared to 9.32 mg/L during the monoculture.

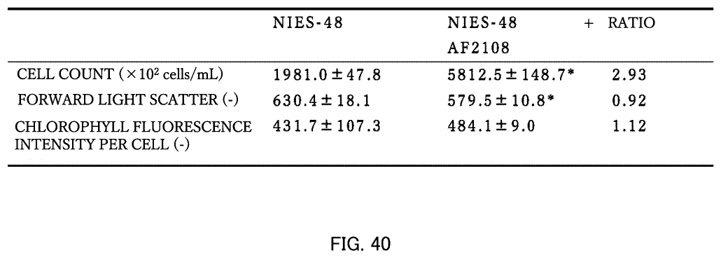

FIG. 40 depicts the results of flow cytometry measurements of the cell count, cell size, and chlorophyll fluorescence intensity of NIES-48 during the monoculture of NIES-48 or during the co-culture with AF2108. The present inventors measured the cell size using forward scatter (FSC-H) in flow cytometry. Marks * in FIG. 40 represent that there is a significant difference from the monoculture of NIES-48 (p<0.05). The cell count after 168 hours of the co-culture of NIES-48 and the AF2108 isolate increased to 2.93 times compared with the monoculture of NIES-48. The chlorophyll fluorescence intensity remained unchanged at 1.12 times. The forward light scatter decreased to 0.92 times. This revealed that the co-culture of NIES-48 and AF2108 promotes the growth, and increases the cell count.

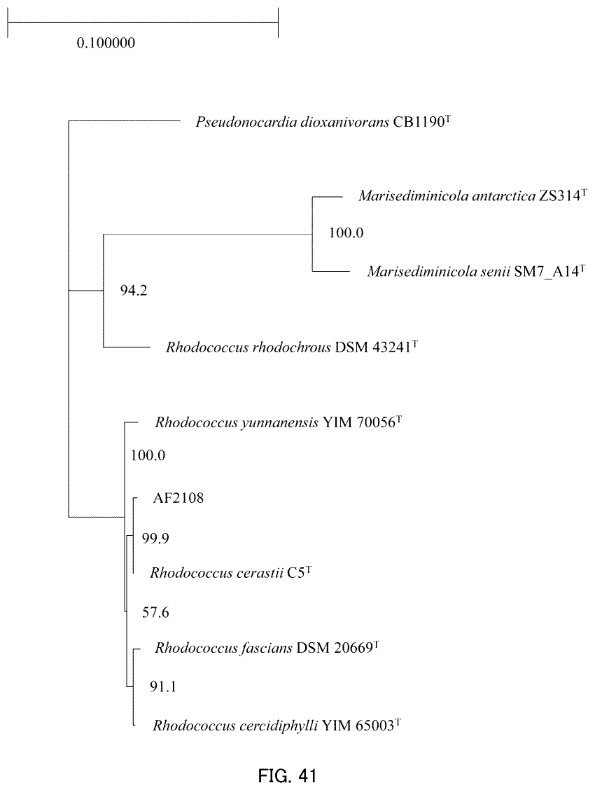

The present inventors determined the almost complete 16S rRNA sequence of the AF2108 strain through sequencing (1394 bp). According to the results of a sequence analysis using BLASTn, the top five strains with high homology were Rhodococcus cerastii C5, Rhodococcus cercidiphylli YIM65003, Rhodococcus yunnanensis YIM 70056, Rhodococcus fascians ATCC 12974, and Rhodococcus fascians CF17, with homology of 100.00%, 99.58%, 99.31%, 99.16%, and 99.15%, respectively. FIG. 41 depicts the phylogenetic tree of Rhodococcus closely related to AF2108. The present inventors constructed the phylogenetic tree depicted in FIG. 41 on the basis of the 16S rRNA gene sequence of AF2108. Bootstrap values which are equal to or greater than 50 are depicted. The Pseudonocardia dioxanivorans CB1190 type strain was used as an outgroup. The present inventors identified AF2108 as Rhodococcus cerastii.

### [JM202 Isolate]

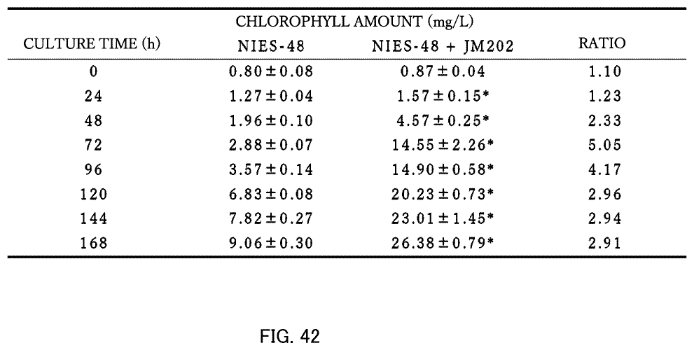

FIG. 42 depicts the measurement results of chlorophyll a+b after the co-culture of the JM202 isolate and NIES-48. Marks * in FIG. 42 represent that there is a significant difference from the monoculture of NIES-48 (p<0.05). By the co-culture of NIES-48 and the JM202 strain by the present inventors, the chlorophyll amount became 26.38 mg/L at 168 hours of culture, and increased to 2.91 times as compared to 9.06 mg/L during the monoculture.

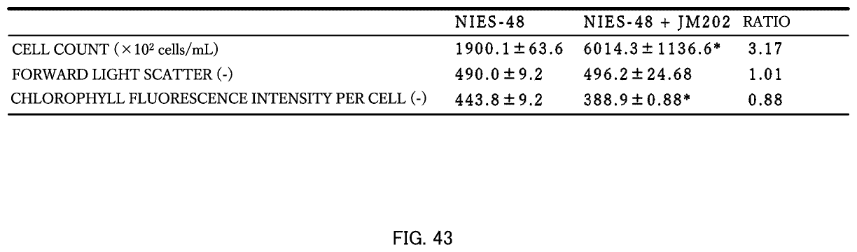

FIG. 43 depicts the results of flow cytometry measurements of the cell count, cell size, and chlorophyll fluorescence intensity of NIES-48 during the monoculture of NIES-48 or during the co-culture with JM202. The present inventors measured the cell size using forward scatter (FSC-H) in flow cytometry. Marks * in FIG. 43 represent that there is a significant difference from the monoculture of NIES-48 (p<0.05). The cell count and chlorophyll fluorescence intensity after 168 hours of the co-culture of NIES-48 and the JM202 isolate were 3.17 times and 0.88 times, respectively, compared with the monoculture of NIES-48. The forward light scatter remained unchanged at 1.01 times. This revealed that the co-culture of NIES-48 and JM202 slightly reduces the chlorophyll amount per cell, but promotes the growth, and increases the cell count.

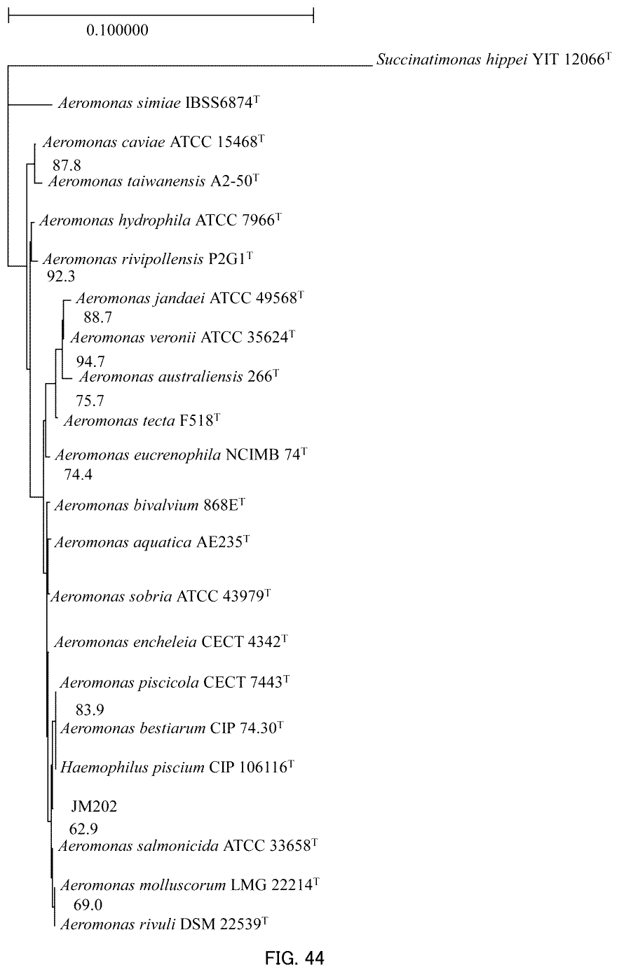

The present inventors determined the almost complete 16S rRNA sequence of the JM202 strain through sequencing (1458 bp). According to the results of a sequence analysis using BLASTn, the top five strains with high homology were Aeromonas salmonicida CECT 894, Aeromonas salmonicida ATCC 33658, Aeromonas salmonicida NCIMB 1102, Aeromonas salmonicida ATCC 33658, and Aeromonas salmonicida subsp. masoucida NBRC 13784, with homology of 99.86%, 99.84%, 99.79%, 99.79%, and 99.79%, respectively. FIG. 44 depicts the phylogenetic tree of Aeromonas closely related to JM202. The present inventors constructed the phylogenetic tree depicted in FIG. 44 on the basis of the 16S rRNA gene sequence of JM202. Bootstrap values which are equal to or greater than 50 are depicted. The present inventors used the Succinatimonas hippei YIT 12066 type strain as an outgroup. JM202 has been deposited in National Institute of Technology and Evaluation, Patent Microorganisms Depositary Center (deposit number: NITE P-03835).

### Effects of Present Invention

The microalgae growth-promoting microorganism belonging to Rhodococcus, Xanthobacter, Ancylobacter, Shewanella sp., Bacillus, or Aeromonas according to the present embodiment can promote the growth of microalgae.

Whereas the present invention has been explained using embodiments thus far, the technical scope of the present embodiments is not limited by the scope described in the embodiments described above, but various modifications and changes are possible within the scope of a gist of the present invention. For example, all or some of apparatuses can be configured functionally or physically distributed or integrated in any units. In addition, new embodiments that are generated by any combination of a plurality of embodiments are also included in embodiments of the present embodiments. Effects of the new embodiments generated by the combination combine effects of the original embodiments.

1. A microalgae growth-promoting microorganism which is a microorganism belonging to Rhodococcus, Xanthobacter, Ancylobacter, Shewanella, or Aeromonas.

2. The microalgae growth-promoting microorganism according to claim 1, wherein the microorganism is Rhodococcus cerastii of Rhodococcus, Xanthobacter flavus of Xanthobacter, or Ancylobacter rudongensis of Ancylobacter.

3. The microalgae growth-promoting microorganism according to claim 1, wherein the microorganism is Rhodococcus cerastii of Rhodococcus, or Aeromonas salmonicida or Aeromonas piscicola of Aeromonas.

4. The microalgae growth-promoting microorganism according to claim 1, wherein the microorganism is for promoting growth of blue-green algae, green algae, glaucophyte algae, or Euglena algae.

5. The microalgae growth-promoting microorganism according to claim 1, wherein the microorganism is for promoting growth of an organism belonging to Phylum Cyanobacteria, Heterokontophyta, Euglena phylum, Cryptophyta, Haptophyta, Cercozoa, Glaucophyta, Rhodophyta, Chlorophyta, or Streptophyta.

6. The microalgae growth-promoting microorganism according to claim 1, wherein the microorganism is for promoting growth of an organism belonging to Phylum Cyanobacteria or Euglenophyta.

7. A microalgae growth-promoting microorganism which is a microorganism belonging to Bacillus for promoting growth Euglena algae.

8. The microalgae growth-promoting microorganism according to claim 7, wherein the microorganism is Bacillus licheniformis, Bacillus pumilus, Bacillus zhangzhouensis, Bacillus australimaris, Bacillus safensis, or Peribacillus acanthi of Bacillus.

9. A microalgae growth promoter containing the microalgae growth-promoting microorganism according to claim 1.

10. A microalgae growth promoter containing a microorganism belonging to Bacillus.
